(19) United States
(12) Patent Application Publication
(10) Pub. No.: US 2024/0182971 A1
(43) Pub. Date: Jun. 6, 2024

(54) METHOD FOR DIAGNOSING DRY MOUTH USING BIOMARKERS

(71) Applicants: Colgate-Palmolive Company, New York, NY (US); The Regents of the University of California, Oakland, CA (US)

(72) Inventors: Bhuvaneswari GURUMURTHY, New York, NY (US); Donghui WU, Bridgewater, NY (US); Michael FITZGERALD, Oakhurst, NJ (US); David WONG, Pasadena, CA (US)

(21) Appl. No.: 18/551,396

(22) PCT Filed: Mar. 18, 2022
(86) PCT No.: PCT/US22/20898

§ 371 Date: Sep. 19, 2023

Related U.S. Application Data
(60) Provisional application No. 63/163,327, filed on Mar. 19, 2021.

(51) Int. Cl. C12Q 1/6883

(52) U.S. Cl. CPC C12Q 1/6883; C12Q 2600/154; C12Q 2600/158

(57) ABSTRACT

The present invention describes a method for the detection and monitoring of xerostomia in a subject using biomarkers.

### BACKGROUND

Dry mouth, clinically called xerostomia, is defined as a subjective feeling of dryness of the mouth. It is caused primarily by reduction of salivary secretion, but the underlying mechanism for such reduction varies from patient to patient. Medication is the most common cause of dry mouth. Medication-induced dry mouth is associated with over 1500 drugs that are either prescribed or available over-the-counter. Polypharmacy—where an individual is taking several drugs at one time is strongly associated with dry mouth: taking at least three medicines per day increases the risk of suffering from dry mouth to around 50%. Other causes include systemic diseases such as Sjögren's syndrome and radiation therapy to the head and neck.

Depending on its severity, dry mouth can cause discomfort and lead to pathological conditions, such as caries and fungal infection, specifically oral candidiasis. Xerostomia is frequent in the elderly. In the geriatric population, xerostomia has been reported to occur in 17 to 39% of the persons aged 65 years or more. In addition, xerostomia is more frequent among women than men. Based on available data, a conservative analysis of the occurrence of xerostomia in the developed world shows a prevalence of 80 million people. However, the far majority are not aware they have the condition. Early detection and diagnosis of xerostomia is important for systemic and oral health maintenance. Thus, it is desirable to develop objective and scientifically credible biomarkers for early detection and monitoring of xerostomia.

It is therefore desirable to develop improved methods for diagnosing and/or treating xerostomia.

### BRIEF SUMMARY

In one aspect, the present invention provides a method of diagnosing xerostomia in a subject, comprising:
(a) isolating a biological sample from the subject;
(b) detecting a level of expression and/or DNA methylation of at least one gene selected from genes listed in Tables 1-4 in the biological sample from said subject;
(c) comparing the level of expression and/or DNA methylation of the at least one gene in the sample to a level of expression in a reference,
   wherein an increased or decreased level of expression and/or DNA methylation of the at least one gene in the sample compared to the level in the reference identifies the subject having xerostomia and wherein the biological sample is biopsied parotid gland or saliva. In some embodiments, the reference is a biological sample of a subject or population not having xerostomia. In some embodiments, the method further comprises a step of treating the subject for xerostomia. In certain embodiments, the biological sample is saliva.

In some embodiments, the at least one gene is selected from 32 genes listed in Tables 1 and 2. In some embodiments, the at least one gene is selected from 14 genes listed in Table 1. In some embodiments, the at least one gene is selected from 18 genes listed in Table 2. In some embodiments, the at least one gene is selected from 97 genes listed in Tables 4 and 5. In some embodiments, the at least one gene is selected from 36 genes listed in Table 4. In some embodiments, the at least one gene is selected from 61 genes listed in Table 5. In some embodiments, the at least one gene is selected from the group consisting of KCNJ10, KCNJ2, PRKCA, PIK3CG, RASSF5, CDS1, IFI30, HLA-B, and B2M. In some embodiments, the at least one gene is selected from the group consisting of KCNJ10 and KCNJ2. In some embodiments, the at least one gene is selected from the group consisting of PRKCA, PIK3CG, RASSF5, CDS1, IFI30, HLA-B, and B2M. In some embodiments, the at least one gene is selected from the group consisting of PRKCA, PIK3CG, RASSF5. In some embodiments, the at least one gene is selected from the group consisting of PRKCA, PIK3CG, CDS1. In some embodiments, the at least one gene is selected from the group consisting of IFI30, HLA-B, and B2M.

In some embodiments, the level of expression of the at least one gene in the biological sample is determined by measuring the level of mRNA of the at least one gene in the biological sample. In some embodiments, the level of expression of the at least one gene in the biological sample is determined by measuring the level of polypeptide of the at least one gene in the biological sample.

In another aspect, the present invention provides a method of monitoring the response to a xerostomia treatment in a subject. The method comprises
(a) isolating a biological sample from the subject after the treatment is initiated;
(b) detecting a level of expression and/or DNA methylation of at least one gene selected from genes listed in Tables 1, 2, 4 and 5 in the biological sample from said subject;
(c) comparing the level of expression and/or DNA methylation of the at least one gene in the sample to a level of expression and/or DNA methylation in a reference,
   wherein an increased or decreased level of expression and/or DNA methylation of the at least one gene in the sample compared to the level in the reference indicates that the subject is responsive to the treatment and wherein the biological sample is biopsied parotid gland or saliva. In some embodiments, the reference is a biological sample of the subject obtained prior to initiation of the treatment. In some embodiments, the reference is a biological sample of the subject obtained at an earlier time point during the treatment. In certain embodiments, the biological sample is saliva.

In another aspect, the present invention provides a method of treating xerostomia, comprising administering a xerostomia treatment to a subject identified as having a differential level of expression and/or differential DNA methylation of at least one gene selected from genes listed in Tables 1, 2, 4 and 5 in a biological sample of the subject, wherein the biological sample is biopsied parotid gland or saliva.

In another aspect, the present invention provides a method of detecting a level of expression and/or DNA methylation of at least one gene selected from genes listed in Tables 1, 2, 4 and 5 in a subject, comprising obtaining a biological sample of a subject and detecting a level of expression (e.g., mRNA or polypeptide) and/or DNA methylation of the at least one gene in the biological sample of the subject, wherein the level of mRNA of the at least one gene is detected by nucleic acid microarrays, quantitative PCR, real time PCR, sequencing (e.g., next generation sequencing), or the level of polypeptide of the at least one gene is detected by ELISA, Western blot, flow cytometry, immunofluorescence, immunohistochemistry, and mass spectroscopy, or the level of DNA methylation of the at least one gene is detected by bisulfite sequencing, methylation specific melting curve analysis (MS-MCA), high resolution melting (MS-HRM), MALDI-TOF MS, methylation specific MLPA, methylated-DNA precipitation/enrichment and methylation-sensitive restriction enzymes (COMPARE-MS), methylation sensitive oligonucleotide microarray, Infinium and MethylLight via antibodies and protein binding domains targeted to methylated DNA or single molecule real time sequencing, Multiplex methylation based PCR assays, Illumina Methylation Assay using ‘BeadChip’ technology, and wherein the biological sample is biopsied parotid gland or saliva.

In another aspect, the present invention provides a kit for diagnosing and/or monitoring xerostomia comprising at least one reagent for the determination of the level of expression and/or DNA methylation of at least one gene selected from genes listed in Tables 1, 2, 4 and 5 in a biological sample selected from biopsied parotid gland or saliva.

In another aspect, the invention provides a method of treating a subject suffering from xerostomia (dry mouth), comprising:
(a) diagnosing xerostomia using the method according to the invention, e.g., any of Method 1, et seq., and
(b) administering a xerostomia treatment to the subject.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### BRIEF DESCRIPTION OF THE DRAWINGS

The present invention will become more fully understood from the detailed description and the accompanying drawings.

FIG. 1 shows Volcano plot of RNA profiling: dry mouth vs. healthy parotid glands.

FIG. 2 shows Principle Component Analysis (PCA) of RNA profiling based on 167 DE (differential expression) probe sets: dry mouth vs. healthy parotid glands.

FIG. 3 shows Volcano plot of DNA methylation: dry mouth vs. healthy parotid glands.

FIG. 4 shows Principle Component Analysis (PCA) of DNA methylation based on 704 DM (differential methylation) CpG sites: dry mouth vs. healthy parotid glands.

FIG. 5 shows Volcano plot of RNA profiling: dry mouth vs. healthy saliva.

FIG. 6 shows Principle Component Analysis (PCA) of RNA profiling based on 299 DE (differential expression) probe sets: dry mouth vs. healthy saliva.

FIG. 7 shows Volcano plot of DNA methylation: dry mouth vs. healthy saliva.

FIG. 8 shows Principle Component Analysis (PCA) of DNA methylation based on 2596 DM (differential methylation) CpG sites: dry mouth vs. healthy saliva.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range.

The present invention relates to methods to detect and measure saliva-based genes for the detection of xerostomia in a subject. For example, in some embodiments, the genes described herein can be used to assess the status of xerostomia, monitor xerostomia regression or monitor a response to xerostomia treatment. The markers of the invention can be used to screen, diagnose and monitor xerostomia. The detection or diagnosis of xerostomia in a subject using the markers of the invention can be used to establish and evaluate treatment plans for xerostomia. Furthermore, the biological pathways and molecular targets/genes identified in the present invention can enable specific targeting for therapeutic interventions of dry mouth.

In an aspect, the present invention provides a method (Method 1.0) of diagnosing xerostomia (i.e., dry mouth) in a subject, comprising:
(a) isolating a biological sample from the subject;
(b) detecting a level of expression and/or DNA methylation of at least one gene selected from genes listed in Tables 1, 2, 4 and 5 in the biological sample from the subject;
(c) comparing the level of expression and/or DNA methylation of the at least one gene in the sample to a level of expression and/or DNA methylation in a reference,
   wherein an increased or decreased level of expression and/or DNA methylation of the at least one gene in the sample compared to the level in the reference identifies the subject having xerostomia and wherein the biological sample is biopsied parotid gland or saliva.

For example, the invention includes:
1.1 Method 1.0, wherein the at least one gene is selected from 32 genes listed in Tables 1 and 2, optionally wherein the biological sample is biopsied parotid gland.
1.2 Method 1.0, wherein the at least one gene is selected from 14 genes listed in Table 1, optionally wherein the biological sample is biopsied parotid gland.
1.3 Method 1.0, wherein the at least one gene is selected from 18 genes listed in Table 2, optionally wherein the biological sample is biopsied parotid gland.
1.4 Method 1.0, wherein the at least one gene is selected from 97 genes listed in Tables 4 and 5, optionally wherein the biological sample is saliva.
1.5 Method 1.0, wherein the at least one gene is selected from 36 genes listed in Table 4, optionally wherein the biological sample is saliva.
1.6 Method 1.0, wherein the at least one gene is selected from 61 genes listed in Tables 5, optionally wherein the biological sample is saliva.
1.7 Method 1.0, wherein the at least one gene is selected from the group consisting of KCNJ10, KCNJ2, PRKCA, PIK3CG, RASSF5, CDS1, IFI30, HLA-B, and B2M.
1.8 Method 1.7, wherein the at least one gene comprises KCNJ10, KCNJ2, PRKCA, PIK3CG, RASSF5, CDS1, IFI30, HLA-B, and B2M.
1.9 Method 1.0, wherein the at least one gene is selected from the group consisting of KCNJ10 and KCNJ2, optionally wherein the biological sample is biopsied parotid gland.
1.10 Method 1.9, wherein the at least one gene comprises KCNJ10 and KCNJ2, optionally wherein the biological sample is biopsied parotid gland.
1.11 Method 1.0, wherein the at least one gene is selected from the group consisting of PRKCA, PIK3CG, RASSF5, CDS1, IFI30, HLA-B, and B2M, optionally wherein the biological sample is saliva.
1.12 Method 1.11, wherein the at least one gene comprises PRKCA, PIK3CG, RASSF5, CDS1, IFI30, HLA-B, and B2M, optionally wherein the biological sample is saliva.
1.13 Method 1.0, wherein the at least one gene is selected from the group consisting of PRKCA, PIK3CG, RASSF5, optionally wherein the biological sample is saliva.
1.14 Method 1.13, wherein the at least one gene comprises PRKCA, PIK3CG, RASSF5, optionally wherein the biological sample is saliva.
1.15 Method 1.0, wherein the at least one gene is selected from the group consisting of PRKCA, PIK3CG, CDS1, optionally wherein the biological sample is saliva.
1.16 Method 1.15, wherein the at least one gene comprises PRKCA, PIK3CG, CDS1, optionally wherein the biological sample is saliva.
1.17 Method 1.0, wherein the at least one gene is selected from the group consisting of IFI30, HLA-B, and B2M, optionally wherein the biological sample is saliva.
1.18 Method 1.17, wherein the at least one gene comprises IFI30, HLA-B, and B2M, optionally wherein the biological sample is saliva.
1.19 Any of the preceding methods, wherein the level of expression of the at least one gene in the biological sample is determined by measuring the level of mRNA of the at least one gene in the biological sample.
1.20 Any of the preceding methods, wherein the level of expression of the at least one gene in the biological sample is determined by measuring the level of polypeptide of the at least one gene in the biological sample.
1.21 Any of the preceding methods, wherein the level of DNA methylation of the at least one gene in the biological sample is determined by measuring the level of DNA methylation at a CpG site located within or near the gene, optionally wherein the CpG site is located in the promoter region of the gene, further optionally wherein the CpG site is located in a CpG island in the promoter region of the gene.
1.22 Any of the preceding methods, wherein the subject has taken one or more medications, optionally wherein the one or more medications are selected from anti-depressants, bronchodilators, anti-hyperlipidemics, anti-hypertensives, analgesics, anti-inflammatory agents, vasodilators, estrogen modulators, eye lubricants, anorectics, antiarrhythmics, anticholinergics, anticonvulsants, antidiarrhoeals, anti-emetics, antihistamines/decongestants, antiparkinsonians, antipsychotics, antispasmodics and diuretics and combinations thereof.
1.23 Any of the preceding methods, wherein the subject is a patient with a condition selected from Sjögren's syndrome, rheumatoid arthritis, systemic lupus erythematosus, scleroderma, mixed connective tissue disease, sarcoidosis, Crohn's disease, ulcerative colitis, celiac disease, autoimmune liver disease, amyloidosis, diabetes mellitus, thyroiditis, Parkinson's disease, burning mouth syndrome, anxiety and depression, narcolepsia, Epstein-Barr virus and cytomegalovirus infections, cystic fibrosis, dehydration, and anorexia nervosa.
1.24 Method 1.23, wherein the subject is a patient with Sjögren's syndrome.
1.25 Any of the preceding methods, wherein the subject has been treated with cancer treatment, e.g., radiation.
1.26 Any of the preceding methods, wherein the reference is a biological sample of a subject or population not having xerostomia.
1.27 Any of the preceding methods, the method further comprises a step of treating the subject for xerostomia, optionally wherein the treatment comprises administering a therapeutic agent (e.g. pilocarpine) that boosts saliva production to the subject, applying an oral care composition containing an agent to treat or alleviate xerostomia or reduce friction between oral surfaces or boost salivary production (e.g., an oral care composition comprising a fluoride ion source, artificial saliva substitute or moisturizers, or a mouthwash such as Colgate® Hydris™ Oral Rinse) to the oral cavity, changing medications that causes xerostomia (e.g., adjusting the dose of medication or switching to a different drug that doesn't cause xerostomia) if the subject has taken medications that causes xerostomia, or a combination thereof.
1.28 Any of the preceding methods, wherein the biological sample is saliva.
1.29 Any of the preceding methods, wherein the biological sample is biopsied parotid gland.
1.30 Any of the preceding methods, wherein the subject is human.

In an aspect, the present invention provides a method (Method 2.0) of monitoring the response to a xerostomia treatment in a subject, comprising
(a) isolating a biological sample from the subject after the treatment is initiated;
(b) detecting a level of expression and/or DNA methylation of at least one gene selected from genes listed in Tables 1, 2, 4, and 5 in the biological sample from the subject;
(c) comparing the level of expression and/or DNA methylation of the at least one gene in the sample to a level of expression and/or DNA methylation in a reference,
   wherein an increased or decreased level of expression and/or DNA methylation of the at least one gene in the sample compared to the level in the reference indicates that the subject is responsive to the treatment and wherein the biological sample is biopsied parotid gland or saliva.

For example, the invention includes:
2.1. Method 2.0, wherein the at least one gene is selected from 32 genes listed in Tables 1 and 2, optionally wherein the biological sample is biopsied parotid gland.
2.2. Method 2.0, wherein the at least one gene is selected from 14 genes listed in Table 1, optionally wherein the biological sample is biopsied parotid gland.
2.3. Method 2.0, wherein the at least one gene is selected from 18 genes listed in Table 2, optionally wherein the biological sample is biopsied parotid gland.
2.4. Method 2.0, wherein the at least one gene is selected from 97 genes listed in Tables 4 and 5, optionally wherein the biological sample is saliva.
2.5. Method 2.0, wherein the at least one gene is selected from 36 genes listed in Table 4, optionally wherein the biological sample is saliva.
2.6. Method 2.0, wherein the at least one gene is selected from 61 genes listed in Tables 5, optionally wherein the biological sample is saliva.
2.7. Method 2.0, wherein the at least one gene is selected from the group consisting of KCNJ10, KCNJ2, PRKCA, PIK3CG, RASSF5, CDS1, IFI30, HLA-B, and B2M.
2.8. Method 2.7, wherein the at least one gene comprises KCNJ10, KCNJ2, PRKCA, PIK3CG, RASSF5, CDS1, IFI30, HLA-B, and B2M.
2.9. Method 2.0, wherein the at least one gene is selected from the group consisting of KCNJ10 and KCNJ2, optionally wherein the biological sample is biopsied parotid gland.
2.10. Method 2.9, wherein the at least one gene comprises KCNJ10 and KCNJ2, optionally wherein the biological sample is biopsied parotid gland.
2.11. Method 2.0, wherein the at least one gene is selected from the group consisting of PRKCA, PIK3CG, RASSF5, CDS1, IFI30, HLA-B, and B2M, optionally wherein the biological sample is saliva.
2.12. Method 2.11, wherein the at least one gene comprises PRKCA, PIK3CG, RASSF5, CDS1, IFI30, HLA-B, and B2M, optionally wherein the biological sample is saliva.
2.13. Method 2.0, wherein the at least one gene is selected from the group consisting of PRKCA, PIK3CG, RASSF5, optionally wherein the biological sample is saliva.
2.14. Method 2.13, wherein the at least one gene comprises PRKCA, PIK3CG, RASSF5, optionally wherein the biological sample is saliva.
2.15. Method 2.0, wherein the at least one gene is selected from the group consisting of PRKCA, PIK3CG, CDS1, optionally wherein the biological sample is saliva.
2.16. Method 2.15, wherein the at least one gene comprises PRKCA, PIK3CG, CDS1, optionally wherein the biological sample is saliva.
2.17. Method 2.0, wherein the at least one gene is selected from the group consisting of IFI30, HLA-B, and B2M, optionally wherein the biological sample is saliva.
2.18. Method 2.17, wherein the at least one gene comprises IFI30, HLA-B, and B2M, optionally wherein the biological sample is saliva.
2.19. Any of the preceding methods, wherein the level of expression of the at least one gene in the biological sample is determined by measuring the level of mRNA of the at least one gene in the biological sample.
2.20. Any of the preceding methods, wherein the level of expression of the at least one gene in the biological sample is determined by measuring the level of polypeptide of the at least one gene in the biological sample.
2.21. Any of the preceding methods, wherein the level of DNA methylation of the at least one gene in the biological sample is determined by measuring the level of DNA methylation at a CpG site located within or near the gene, optionally wherein the CpG site is located in the promoter region of the gene, further optionally wherein the CpG site is located in a CpG island in the promoter region of the gene
2.22. Any of the preceding methods, wherein the subject has taken one or more medications, optionally wherein the one or more medications are selected from anti-depressants, bronchodilators, anti-hyperlipidemics, anti-hypertensives, analgesics, anti-inflammatory agents, vasodilators, estrogen modulators, eye lubricants, anorectics, antiarrhythmics, anticholinergics, anticonvulsants, antidiarrhoeals, anti-emetics, antihistamines/decongestants, antiparkinsonians, antipsychotics, antispasmodics and diuretics and combinations thereof.
2.23. Any of the preceding methods, wherein the subject is a patient with a condition selected from Sjögren's syndrome, rheumatoid arthritis, systemic lupus erythematosus, scleroderma, mixed connective tissue disease, sarcoidosis, Crohn's disease, ulcerative colitis, celiac disease, autoimmune liver disease, amyloidosis, diabetes mellitus, thyroiditis, Parkinson's disease, burning mouth syndrome, anxiety and depression, narcolepsia, Epstein-Barr virus and cytomegalovirus infections, cystic fibrosis, dehydration, and anorexia nervosa.
2.24. Method 2.23, wherein the subject is a patient with Sjögren's syndrome.
2.25. Any of the preceding methods, wherein the subject has been treated with cancer treatment, e.g., radiation.
2.26. Any of the preceding methods, wherein the reference is a biological sample of the subject obtained prior to initiation of the treatment or the reference is a biological sample of the subject obtained at an earlier time point during the treatment.
2.27. Any of the preceding methods, wherein the biological sample is saliva.
2.28. Any of the preceding methods, wherein the biological sample is biopsied parotid gland.
2.29. Any of the preceding methods, wherein the subject is human.

In an aspect, the present invention provides a method (Method 3.0) of detecting a level of expression and/or DNA methylation of at least one gene selected from genes listed in Tables 1, 2, 4, and 5 in a subject, comprising obtaining a biological sample of a subject and detecting a level of expression (e.g., mRNA or polypeptide) and/or DNA methylation of the at least one gene in the biological sample of the subject, wherein the biological sample is biopsied parotid gland or saliva.

For example, the invention includes:
3.1. Method 3.0, wherein the at least one gene is selected from 32 genes listed in Tables 1 and 2, optionally wherein the biological sample is biopsied parotid gland.
3.2. Method 3.0, wherein the at least one gene is selected from 14 genes listed in Table 1, optionally wherein the biological sample is biopsied parotid gland.
3.3. Method 3.0, wherein the at least one gene is selected from 18 genes listed in Table 2, optionally wherein the biological sample is biopsied parotid gland.
3.4. Method 3.0, wherein the at least one gene is selected from 97 genes listed in Tables 4 and 5, optionally wherein the biological sample is saliva.
3.5. Method 3.0, wherein the at least one gene is selected from 36 genes listed in Table 4, optionally wherein the biological sample is saliva.
3.6 Method 3.0, wherein the at least one gene is selected from 61 genes listed in Tables 5, optionally wherein the biological sample is saliva.
3.7. Method 3.0, wherein the at least one gene is selected from the group consisting of KCNJ10, KCNJ2, PRKCA, PIK3CG, RASSF5, CDS1, IFI30, HLA-B, and B2M.
3.8. Method 3.7, wherein the at least one gene comprises KCNJ10, KCNJ2, PRKCA, PIK3CG, RASSF5, CDS1, IFI30, HLA-B, and B2M.
3.9. Method 3.0, wherein the at least one gene is selected from the group consisting of KCNJ10 and KCNJ2, optionally wherein the biological sample is biopsied parotid gland.
3.10. Method 3.9, wherein the at least one gene comprises KCNJ10 and KCNJ2, optionally wherein the biological sample is biopsied parotid gland.
3.11. Method 3.0, wherein the at least one gene is selected from the group consisting of PRKCA, PIK3CG, RASSF5, CDS1, IFI30, HLA-B, and B2M, optionally wherein the biological sample is saliva.
3.12. Method 3.11, wherein the at least one gene comprises PRKCA, PIK3CG, RASSF5, CDS1, IFI30, HLA-B, and B2M, optionally wherein the biological sample is saliva.
3.13. Method 3.0, wherein the at least one gene is selected from the group consisting of PRKCA, PIK3CG, RASSF5, optionally wherein the biological sample is saliva.
3.14. Method 3.13, wherein the at least one gene comprises PRKCA, PIK3CG, RASSF5, optionally wherein the biological sample is saliva.
3.15. Method 3.0, wherein the at least one gene is selected from the group consisting of PRKCA, PIK3CG, CDS1, optionally wherein the biological sample is saliva.
3.16. Method 3.15, wherein the at least one gene comprises PRKCA, PIK3CG, CDS1, optionally wherein the biological sample is saliva.
3.17. Method 3.0, wherein the at least one gene is selected from the group consisting of IFI30, HLA-B, and B2M, optionally wherein the biological sample is saliva.
3.18. Method 3.17, wherein the at least one gene comprises IFI30, HLA-B, and B2M, optionally wherein the biological sample is saliva.
3.19. Any of the preceding methods, wherein the subject has taken one or more medications, optionally wherein the one or more medications are selected from anti-depressants, bronchodilators, anti-hyperlipidemics, anti-hypertensives, analgesics, anti-inflammatory agents, vasodilators, estrogen modulators, eye lubricants, anorectics, antiarrhythmics, anticholinergics, anticonvulsants, antidiarrhoeals, anti-emetics, antihistamines/decongestants, antiparkinsonians, antipsychotics, antispasmodics and diuretics and combinations thereof.
3.20. Any of the preceding methods, wherein the subject is a patient with a condition selected from Sjögren's syndrome, rheumatoid arthritis, systemic lupus erythematosus, scleroderma, mixed connective tissue disease, sarcoidosis, Crohn's disease, ulcerative colitis, celiac disease, autoimmune liver disease, amyloidosis, diabetes mellitus, thyroiditis, Parkinson's disease, burning mouth syndrome, anxiety and depression, narcolepsia, Epstein-Barr virus and cytomegalovirus infections, cystic fibrosis, dehydration, and anorexia nervosa.
3.21. Method 3.20, wherein the subject is a patient with Sjögren's syndrome.
3.22. Any of the preceding methods, wherein the level of mRNA of the at least one gene is detected by nucleic acid microarrays, quantitative PCR, real time PCR, sequencing (e.g., next generation sequencing).
3.23. Any of Methods 3.0-3.21, wherein the level of polypeptide of the at least one gene is detected by ELISA, Western blot, flow cytometry, immunofluorescence, immunohistochemistry, and mass spectroscopy.
3.24. Any of Methods 3.0-3.21, wherein the level of DNA methylation of the at least one gene is detected by bisulfite sequencing, methylation specific melting curve analysis (MS-MCA), high resolution melting (MS-HRM), MALDI-TOF MS, methylation specific MLPA, methylated-DNA precipitation/enrichment and methylation-sensitive restriction enzymes (COMPARE-MS), methylation sensitive oligonucleotide microarray, Infinium and MethylLight via antibodies and protein binding domains targeted to methylated DNA or single molecule real time sequencing, Multiplex methylation based PCR assays, Illumina Methylation Assay using ‘BeadChip’ technology.
3.25. Method 3.24, wherein the level of DNA methylation of the at least one gene in the biological sample is detected by detecting the level of DNA methylation at a CpG site located within or near the gene, optionally wherein the CpG site is located in the promoter region of the gene, further optionally wherein the CpG site is located in a CpG island in the promoter region of the gene.
3.26. Any of the preceding methods, wherein the biological sample is saliva.
3.27. Any of the preceding methods, wherein the biological sample is biopsied parotid gland.

The present invention provides methods of diagnosing and monitoring xerostomia by examining expression and DNA methylation of relevant genes. In some embodiments, the genes for the detection of xerostomia or for monitoring of xerostomia regression or response to treatment include but are not limited to genes listed in Tables 1, 2, 4, and 5. In some embodiments, the genes include but are not limited to 32 genes listed in Tables 1 and 2. In some embodiments, the genes include but are not limited to 14 genes listed in Table 1. In some embodiments, the genes include but are not limited to 18 genes listed in Table 2. In some embodiments, the genes include but are not limited to 97 genes listed in Tables 4 and 5. In some embodiments, the genes include but are not limited to 36 genes listed in Table 4. In some embodiments, the genes include but are not limited to 61 genes listed in Table 5.

In some embodiments, the genes include but are not limited to KCNJ10, KCNJ2, PRKCA, PIK3CG, RASSF5, CDS1, IFI30, HLA-B, and B2M. In some embodiments, the genes include but are not limited to KCNJ10 and KCNJ2. In some embodiments, the genes include but are not limited to PRKCA, PIK3CG, RASSF5, CDS1, IFI30, HLA-B, and B2M. In some embodiments, the genes include but are not limited to PRKCA, PIK3CG, RASSF5. In some embodiments, the genes include but are not limited to PRKCA, PIK3CG, CDS1. In some embodiments, the genes include but are not limited to IFI30, HLA-B, and B2M.

“Sample” as used herein means a biological material isolated from an individual. The biological sample may contain any biological material suitable for detecting the desired biomarkers, and may comprise cellular and/or non-cellular material obtained from the individual. One example of a biological sample is a whole saliva sample. Another example of a biological sample is a cell-free saliva sample. Another example of a biological sample is a saliva supernatant, such as the supernatant obtained after centrifuging a saliva sample. Another example of a biological sample is the material in a pellet obtained from a saliva sample, such as a pellet obtained after centrifuging a saliva sample (i.e., saliva pellet). In some embodiments, the saliva sample is a whole saliva sample. Another example of a biological sample is biopsied parotid gland.

The “reference” may be suitable control sample such as for example a sample from a normal, healthy subject having no xerostomia (dry mouth) symptoms and being age-matched to the patient to be diagnosed with the method of the present invention. The reference may be a standardized sample, e.g., a sample comprising material or data from several samples of healthy subjects who have no xerostomia (dry mouth) symptoms. For a method of monitoring the response to a xerostomia treatment, the reference may be a sample of the subject obtained prior to initiation of the treatment or may be a sample of the subject obtained at an earlier time point during the treatment.

The “level” of a biomarker means the absolute amount or relative amount or concentration of the biomarker in the sample. “Increased level of expression and/or DNA methylation” refers to biomarker levels which are increased by at least 10% or more, for example, 20%, 30%, 40%, or 50%, 60%, 70%, 80%, 90% or more, and/or 1.1 fold, 1.2 fold, 1.3 fold, 1.4 fold, 1.5 fold, 1.6 fold, 1.7 fold, 1.8 fold, 1.9 fold, 2.0 fold or more, and any and all whole or partial increments therebetween than a control. “Decreased level of expression and/or DNA methylation” refers to biomarker product levels which are reduced or decreased by at least 10% or more, for example, 20%, 30%, 40%, or 50%, 60%, 70%, 80%, 90% or more, and/or 2.0 fold, 1.9 fold, 1.8 fold, 1.7 fold, 1.6 fold, 1.5 fold, 1.4 fold, 1.3 fold, 1.2 fold, 1.1 fold or more, and any and all whole or partial increments therebetween than a control.

In some embodiments, xerostomia is diagnosed by measuring a level of expression of genes disclosed herein in a biological sample of a subject and comparing it to a level of expression in a reference. The level of expression of gene may be determined by measuring the level of mRNA and/or polypeptide of the gene.

In some embodiments, the level of expression of the at least one gene in the biological sample is determined by measuring the level of mRNA of the at least one gene in the biological sample. The level of mRNA of genes may be determined by any technology known by a man skilled in the art. The measure may be carried out directly on an extracted RNA sample or on retrotranscribed complementary DNA (cDNA) prepared from extracted RNA by technologies well-known in the art. From the RNA or cDNA sample, the amount of nucleic acid transcripts may be measured using any technology known by a man skilled in the art, including nucleic acid microarrays, quantitative PCR, sequencing (e.g., next generation sequencing).

In some embodiments, the level of mRNA is determined using sequencing, e.g., next generation sequencing. Sequencing may be carried out after converting extracted RNA to cDNA using reverse transcriptase or RNA molecules may be directly sequenced. In a particular embodiment, which should not be considered as limiting the scope of the invention, the measurement of the expression level using next generation sequencing may be performed as follows. Briefly, RNA is extracted from a sample (e.g., saliva). After removing rRNA, RNA samples are then reverse transcribed into cDNA. To ensure strand specificity, single stranded cDNA is first synthesized using Super-Script II reverse transcriptase and random primers in the presence of Actinomycin D, and then converted to double stranded cDNA with the second strand marking mix that incorporates dUTP in place of dTTP. Resulting blunt ended cDNA are purified using AMPure XP magnetic beads. After a 3′end adenylation step, adaptor is attached to cDNA. So obtained cDNA (sequencing library) may be amplified by PCR. The sequencing libraries can be sequenced by any next generation sequencing technology known by a man skilled in the art.

In some embodiments, the measurement of the level of mRNA, e.g., by sequencing (e.g., next generation sequencing), is facilitated by capturing and enriching nucleic acids (RNA or cDNA) corresponding to mRNA of interest prior to the measurement. As used herein, enrichment refers to increasing the percentage of the nucleic acids of interest in the sample relative to the initial sample by selectively purifying the nucleic acids of interest. The enrichment of nucleic acids corresponding to mRNA of interest can be carried out on extracted RNA sample or cDNA sample prepared from extracted RNA. In some embodiments, nucleic acids corresponding to mRNA of interest are captured and enriched by hybridizing RNA or cDNA sample to oligonucleotide probes specific for mRNA of interest (e.g., oligonucleotide probes comprising a sequence complementary to a region of mRNA of interest) under conditions allowing for hybridization of the probes and target nucleic acids to form probe-target nucleic acid complexes. Probes may be DNA or RNA, preferably DNA. The length of probes specific for mRNA may be from 30 to 80 nucleotides, e.g., from 40 to 70, from 40 to 60, or about 50 nucleotides. The probe-target nucleic acid complexes can be purified by any technology known by a man skilled in the art. In a preferred embodiment, probes are biotinylated. The biotinylated probe-target nucleic acid complexes can be purified by using a streptavidin-coated substrate, e.g., a streptavidin-coated magnetic particle, e.g., T1 streptavidin coated magnetic bead.

In some embodiments, the level of mRNA may be determined using quantitative PCR. Quantitative, or real-time, PCR is a well known and easily available technology for those skilled in the art and does not need a precise description. In a particular embodiment, which should not be considered as limiting the scope of the invention, the determination of the expression profile using quantitative PCR may be performed as follows. Briefly, the real-time PCR reactions are carried out using the TaqMan Universal PCR Master Mix (Applied Biosystems). 6 μl cDNA is added to a 9 μl PCR mixture containing 7.5 μl TaqMan Universal PCR Master Mix, 0.75 μl of a 20× mixture of probe and primers and 0.75 μl water. The reaction consists of one initiating step of 2 min at 50 deg. C., followed by 10 min at 95 deg. C., and 40 cycles of amplification including 15 sec at 95 deg. C. and 1 min at 60 deg. C. The reaction and data acquisition can be performed using the ABI 7900HT Fast Real-Time PCR System (Applied Biosystems). The number of template transcript molecules in a sample is determined by recording the amplification cycle in the exponential phase (cycle threshold or C_{Q} or C_{T}), at which time the fluorescence signal can be detected above background fluorescence. Thus, the starting number of template transcript molecules is inversely related to C_{T}.

In some embodiments, the level of mRNA may be determined by the use of a nucleic acid microarray. A nucleic acid microarray consists of different nucleic acid probes that are attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes can be nucleic acids such as cDNAs (“cDNA microarray”) or oligonucleotides (“oligonucleotide microarray”). To determine the expression profile of a target nucleic acid sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art.

In some embodiments, the level of expression of the at least one gene in the biological sample is determined by measuring the level of polypeptide of the at least one gene in the biological sample. The level of polypeptide may be determined by any technology known by a man skilled in the art, including ELISA, Western blot, flow cytometry, immunofluorescence, immunohistochemistry, and mass spectroscopy. In particular, the expression level of polypeptide may be determined by using immunodetection methods consisting of using monoclonal antibodies specifically directed against the targeted polypeptides. In some embodiments, the level of polypeptide is determined by measuring fluorescence signal.

In some embodiments, xerostomia is diagnosed by measuring a level of DNA methylation of genes disclosed herein in a biological sample of a subject and comparing it to a level of expression in a reference. The term “DNA Methylation” as disclosed herein includes methylation of any base in DNA. DNA methylation is a biological process by which methyl groups are added to the DNA molecule. Methylation can change the activity of a DNA segment without changing the sequence. When located in a gene promoter, DNA methylation typically acts to repress gene transcription. Two of four bases, cytosine and adenine, can be methylated. Cytosine methylation is widespread in both eukaryotes and prokaryotes, while Adenine methylation has been observed in bacterial, plant, and recently in mammalian DNA, but has received considerably less attention. In mammals, DNA methylation is almost exclusively found in CpG dinucleotides where a cytosine nucleotide is followed by a guanine nucleotide in the linear sequence of bases along its 5′→3′ direction. Cytosines in CpG dinucleotides can be methylated to form 5-methylcytosines. Enzymes that add a methyl group are called DNA methyltransferases. CpG dinucleotides frequently occur in CpG islands. CpG islands are regions with a high frequency of CpG sites. Though objective definitions for CpG islands are limited, the usual formal definition is a region with at least 200 bp, a GC percentage greater than 50%, and an observed-to-expected CpG ratio greater than 60%. Many genes in mammalian genomes have CpG islands associated with the start of the gene (promoter regions). Methylation of the cytosines in CpG sites within a gene can change its expression.

In some embodiments, the level of DNA methylation of the at least one gene in the biological sample is determined by measuring the level of DNA methylation at a CpG site located within or near the gene. In some embodiments, the CpG site is located in the promoter region of the gene. In some embodiments, the CpG site is located in a CpG island in the promoter region of the gene

The level of DNA methylation may be determined by any technology known by a man skilled in the art, including bisulfite sequencing, methylation specific melting curve analysis (MS-MCA), high resolution melting (MS-HRM), MALDI-TOF MS, methylation specific MLPA, methylated-DNA precipitation/enrichment and methylation-sensitive restriction enzymes (COMPARE-MS) or methylation sensitive oligonucleotide microarray, Infinium and MethylLight via antibodies and protein binding domains targeted to methylated DNA as well as single molecule real time sequencing. Multiplex methylation based PCR assays, Illumina Methylation Assay using ‘BeadChip’ technology.

In some embodiments, the level of DNA methylation may be determined by Illumina Methylation Assay using ‘BeadChip’ technology. In a particular embodiment, which should not be considered as limiting the scope of the invention, the determination of the DNA methylation profile using ‘BeadChip’ technology may be performed as follows. Briefly, genomic DNA extracted from a biological sample (e.g., saliva) is used in bisulfite conversion to convert the unmethylated cytosine into uracil. The product contains unconverted cytosine where they were previously methylated, but cytosine converted to uracil if they were previously unmethylated. The bisulfite treated DNA is subjected to whole-genome amplification (WGA) via random hexamer priming and Phi29 DNA polymerase, which has a proofreading activity resulting in error rates 100 times lower than the Taq polymerase. The products are then enzymatically fragmented, purified from dNTPs, primers and enzymes, and applied to the chip. On the chip, there are two bead types for each CpG site per locus. Each locus tested is differentiated by different bead types. Both bead types are attached to single-stranded 50-mer DNA oligonucleotides that differ in sequence only at the free end; this type of probe is known as an allele-specific oligonucleotide. One of the bead types corresponds to the methylated cytosine locus and the other corresponds to the unmethylated cytosine locus, which has been converted into uracil during bisulfite treatment and later amplified as thymine during whole-genome amplification. The bisulfite-converted amplified DNA products are denatured into single strands and hybridized to the chip via allele-specific annealing to either the methylation-specific probe or the non-methylation probe. Hybridization is followed by single-base extension with hapten-labeled dideoxynucleotides. The ddCTP and ddGTP are labeled with biotin while ddATP and ddUTP are labeled with 2,4-dinitrophenol (DNP). After incorporation of these hapten-labeled ddNTPs, multi-layered immunohistochemical assays are performed by repeated rounds of staining with a combination of antibodies to differentiate the two types. After staining, the chip is scanned to show the intensities of the unmethylated and methylated bead types.

In an aspect, the invention provides a kit (Kit 4.0) for diagnosing and/or monitoring xerostomia (dry mouth), comprising at least one reagent for the determination of the level of mRNA or polypeptide or the level of DNA methylation of at least one gene selected from genes listed in Tables 1, 2, 4, and 5.

For example, the invention includes:
4.1. Kit 4.0, wherein the at least one gene is selected from 32 genes listed in Tables 1 and 2.
4.2. Kit 4.0, wherein the at least one gene is selected from 14 genes listed in Table 1.
4.3. Kit 4.0, wherein the at least one gene is selected from 18 genes listed in Table 2.
4.4. Kit 4.0, wherein the at least one gene is selected from 97 genes listed in Tables 4 and 5.
4.5. Kit 4.0, wherein the at least one gene is selected from 36 genes listed in Table 4.
4.6. Kit 4.0, wherein the at least one gene is selected from 61 genes listed in Tables 5.
4.7. Kit 4.0, wherein the at least one gene is selected from the group consisting of KCNJ10, KCNJ2, PRKCA, PIK3CG, RASSF5, CDS1, IFI30, HLA-B, and B2M.
4.8. Kit 4.3, wherein the at least one gene comprises KCNJ10, KCNJ2, PRKCA, PIK3CG, RASSF5, CDS1, IFI30, HLA-B, and B2M.
4.9. Kit 4.0, wherein the at least one gene is selected from the group consisting of KCNJ10 and KCNJ2.
4.10. Kit 4.5, wherein the at least one gene comprises KCNJ10 and KCNJ2.
4.11. Kit 4.0, wherein the at least one gene is selected from the group consisting of PRKCA, PIK3CG, RASSF5, CDS1, IFI30, HLA-B, and B2M.
4.12. Kit 4.7, wherein the at least one gene comprises PRKCA, PIK3CG, RASSF5, CDS1, IFI30, HLA-B, and B2M.
4.13. Kit 4.0, wherein the at least one gene is selected from the group consisting of PRKCA, PIK3CG, RASSF5.
4.14. Kit 4.9, wherein the at least one gene comprises PRKCA, PIK3CG, RASSF5.
4.15. Kit 4.0, wherein the at least one gene is selected from the group consisting of PRKCA, PIK3CG, CDS1.
4.16. Kit 4.11, wherein the at least one gene comprises PRKCA, PIK3CG, CDS1.
4.17. Kit 4.0, wherein the at least one gene is selected from the group consisting of IFI30, HLA-B, and B2M.
4.18. Kit 4.13, wherein the at least one gene comprises IFI30, HLA-B, and B2M.
4.19. Any of the preceding kits, wherein the kit comprises at least one reagent for the determination of the level of mRNA of the at least one gene.
4.20. Kit 4.19, wherein the at least one reagent comprises amplification primer pairs (forward and reverse) and/or probes specific for the mRNA of interest.
4.21. Any of Kits 4.0-4.18, wherein the kit comprises at least one reagent for the determination of the level of polypeptide of the at least one gene.
4.22. Kit 4.21, wherein the at least one reagent comprises monoclonal antibodies specific for the polypeptide of interest.
4.23. Any of Kits 4.0-4.18, wherein the kit comprises at least one reagent for the determination of the level of DNA methylation of the at least one gene.
4.24. Kit 4.23, wherein the at least one reagent comprises a pair of oligonucleotides (e.g., oligonucleotides attached to two different bead types) specific for the methylated and unmethylated DNA site (e.g., CpG site) of interest, respectively.
4.25. Kit 4.24, wherein the DNA site is a CpG site located within or near the gene, optionally wherein the CpG site is located in the promoter region of the gene, further optionally wherein the CpG site is located in a CpG island in the promoter region of the gene.

The term “reagent” means a reagent which specifically allows the determination of the expression or DNA methylation profile, i.e., a reagent specifically intended for the specific determination of the level of mRNA or polypeptide or the level of DNA methylation of gene of interest. Examples include e.g., amplification primer pairs (forward and reward) and/or probes specific for the mRNA of interest, monoclonal antibodies specific for the polypeptide of interest, and a pair of oligonucleotides (e.g., oligonucleotides attached to two different bead types) specific for the methylated and unmethylated DNA site (e.g., CpG site) of interest, respectively. This definition excludes generic reagents useful for the determination of the expression level of DNA methylation level of any other genes that are not disclosed in this disclosure.

In an aspect, the invention provides a method of treating a subject suffering from xerostomia (dry mouth), comprising:
(a) diagnosing xerostomia using the method according to the invention, e.g., any of Method 1, et seq., and
(b) administering a xerostomia treatment to the subject.

Xerostomia may be treated by any treatment known in the art. In some embodiments, the treatment comprises administering a therapeutic agent (e.g. pilocarpine) that boosts saliva production to the subject, applying an oral care composition containing an agent to treat or alleviate xerostomia or reduce friction between oral surfaces or boost salivary production (e.g., an oral care composition comprising a fluoride ion source, artificial saliva substitute or moisturizers, or a mouthwash such as Colgate® Hydris™ Oral Rinse) to the oral cavity, changing medications that causes xerostomia (e.g., adjusting the dose of medication or switching to a different drug that doesn't cause xerostomia) if the subject has taken medications that causes xerostomia, or a combination thereof.

By “treating” or “treatment” of a subject having xerostomia is meant administering or administration of a regimen to the subject in need thereof such that at least one symptom of xerostomia is cured, alleviated, remedied or improved. Examples of therapeutic treatment of xerostomia include, but is not limited to administration of a therapeutic agent (e.g. pilocarpine) that boosts saliva production to the subject, applying an oral care composition containing an agent to treat or alleviate xerostomia or reduce friction between oral surfaces or boost salivary production (e.g., an oral care composition comprising a fluoride ion source, artificial saliva substitute or moisturizers, or a mouthwash such as Colgate® Hydris™ Oral Rinse) to the oral cavity, and changing medications that causes xerostomia (e.g., adjusting the dose of medication or switching to a different drug that doesn't cause xerostomia) if the subject has taken medications that causes xerostomia. In some embodiments, the xerostomia treatment is acupuncture or intraoral electrical stimulation.

Targeted treatment can be achieved by modulating the effects of some of differentially expressed genes in a subject suffering from dry mouth, e.g., a patient with Sjögren's syndrome. For example, seletasilib was tested as an investigative drug for the treatment of Sjögren's syndrome in a mouse model of focal sialadenitis. The drug improved the saliva production, lowered the level of autoantibodies and inflammatory mediators and reduced the immune cell infiltration of salivary glands by inhibiting PI3K delta isoform of phosphatidylinositol 3-kinase delta pathway (Nayar et al. Ann Rheum Dis. 2019; 78:249-60). This pathway is related to the phosphatidylinositol pathway. Biological processes related to immune response are predominantly enriched and is in concordance with the current understanding of salivary pathophysiology in Sjögren's syndrome. Anti-B cell therapies are being explored to decrease the antigen presentation by B-cells for the management of Sjögren's syndrome (Both T et al. Int J Med Sci 2017; 14:191-200).

### EXAMPLES

20 dry mouth parotid glands and saliva and 20 normal parotid glands and saliva were used in this study. 20 dry mouth subjects were non-Sjögren's, non-radiation induced dry mouth patients. 20 normal subjects were matched to dry mouth subjects for age, gender, smoking history and ethnicity. The saliva and parotid gland samples were molecularly profiled by RNA transcriptome analysis using RNA microarrays and DNA methylation analyses in order to identify salivary biomarkers that can reflect dry mouth for clinical evaluation as well as a non-invasive biofluid for early detection of this clinical condition.

### RNA Profiling and DNA Methylation in Parotid Glands

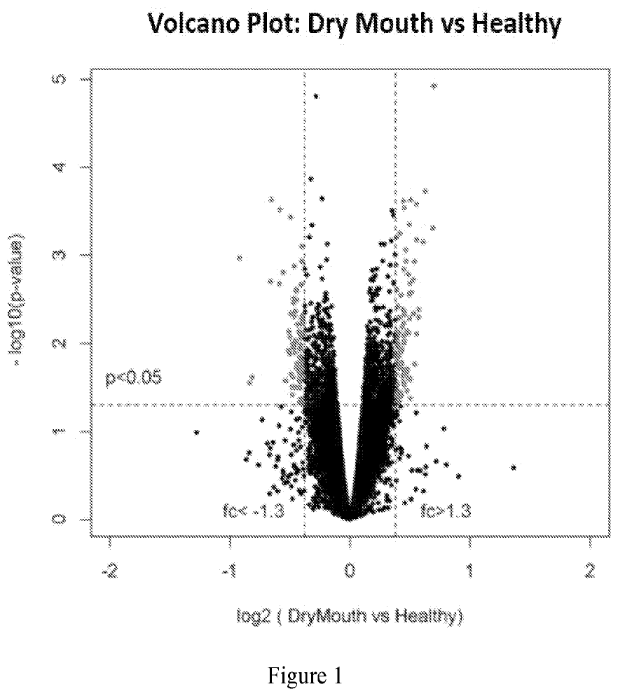

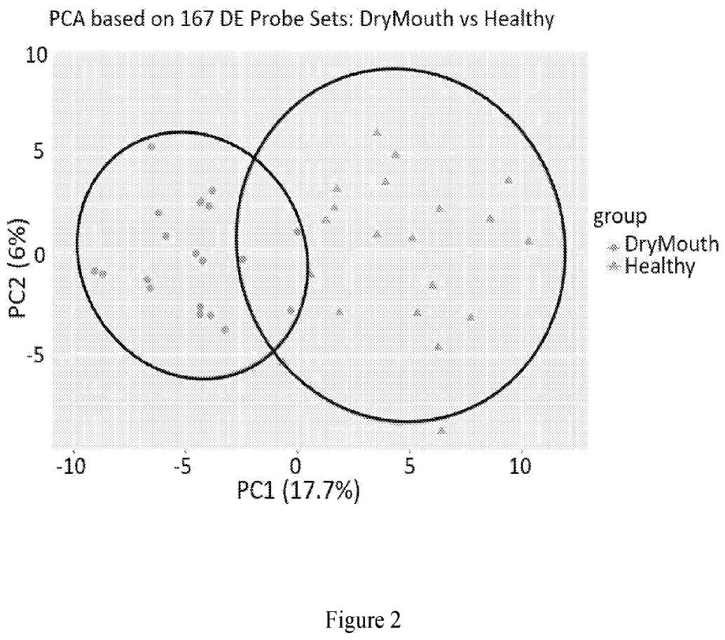

For RNA profiling, RNA was extracted from the parotid glands and quality of the extracted RNA was analyzed by Agilent Bioanalyzer using the RNA 6000 Pico kit as well as the Quant-iTribogreen RNA assay. All 20 healthy and 20 dry mouth parotid gland samples showed excellent quality and quantity RNA as revealed by the presence of intact 18S and 28S rRNA as well as total RNA yield of >5 ng. The extracted RNA from healthy parotid glands and dry mouth parotid glands were constructed for long and small RNA libraries, for a total of 40 libraries. The quality of the RNA libraries were excellent as revealed by long RNA library showing major peak at 300-400 bp whereas small RNA library showing major peak at 140-200 bp. RNA quantity as shown by Qubit dsDNA BR assay revealed concentration >10 nm in each sample. The 40 RNA libraries were profiled using the GeneChip Human Transcriptome Affymetrix HTA 2.0 expression arrays. For the analysis of Affymetrix GeneChip HTA 2.0 RNA expression datasets, the Robust Multi-Array Average (RMA) method was applied for background correction. Data were normalized with quantile normalization and Tukey's Median Polish Approach was used to summarize probe intensities. In this step, the measured signal intensities of >6 million probes were summarized into gene level probe sets (n=70523). ComBat method was applied to remove the batch effects of microarrays. We selected probe sets meeting the following criteria:
1) More than 20% arrays have expression index (log2 scale) of at least 5. This step eliminates probes with low expression index;
2) Coefficient of variation is greater than 0.1 across all arrays. This step excludes probes with low variability.
   Using these criteria, 23,111 out of 70,523 probes remained after filtering. Bioconductor package LIMMA (linear models for microarray data) was used for differential gene expression analysis. Out of 23,111 probes, 167 probes showed >1.3 fold or <−1.3 fold differential expression and were significant by LIMMA's moderated t-test (p<0.05) between dry mouth subjects (n=20) and healthy subjects (n=20). 88 genes were upregulated and 79 genes were downregulated in dry mouth parotid gland. Volcano plot is shown in FIG. 1. Principal component analysis (PCA) plot was used to visualize the separation of the two groups based on expression profiles of the 167 probes (FIG. 2).

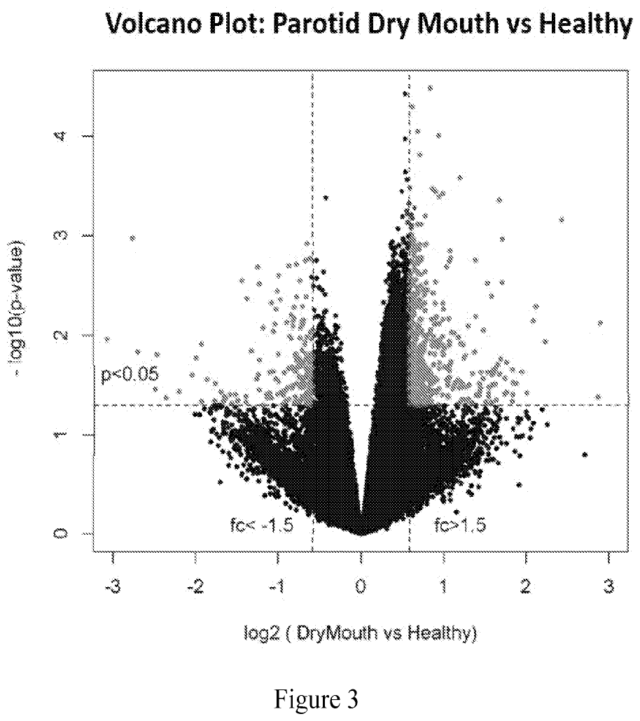

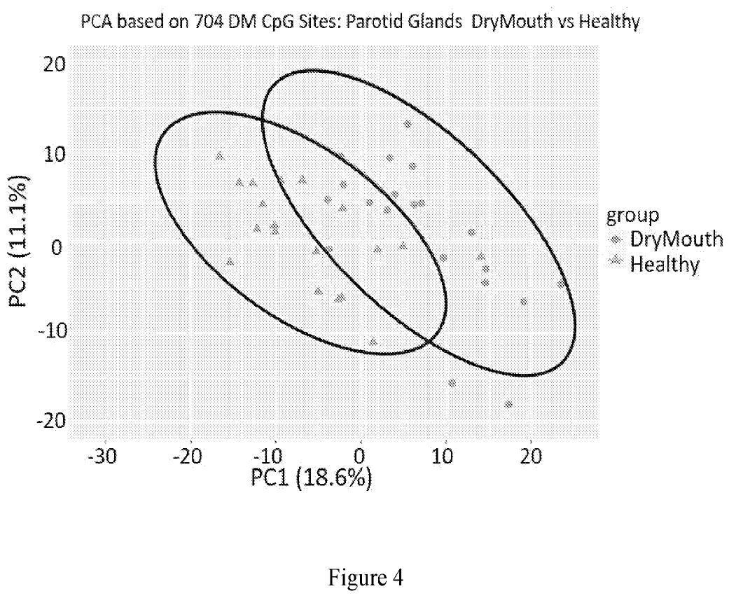

For DNA methylation profiling. DNA was extracted from parotid glands of 20 healthy and 20 dry mouth subjects using the commercial PureLink™ Genomic DNA Mini Kit (Life Technologies, Grand Island NY). The concentration of DNA was measured by NanoDrop® ND-1000 Spectrophotometer (Thermo Scientific). The quality of extracted DNA was evaluated by PCR amplification of the housekeeping gene GAPDH (forward primer: TGGTCTGAGGTCTGAGGTTAAAT; reverse primer: TAGTCCCAGGGCTTTGATTTGC). Quality control of the genomic DNA extracted from healthy and dry mouth parotid glands were all satisfactory as evidenced by the amplification of the 177-bp GAPDH amplicon. Genomic DNAs from healthy and dry mouth parotid glands were comprehensively profiled using the Illumina human methylation 450K bead chip type2 design probes. For the analysis of Illumina Infinium 450 k DNA methylation datasets, Beta-Mixture Quantile Dilation (BMIQ) Normalization method was applied. This is an intra-sample normalization technique aimed to adjust the beta-values of Illumina human methylation 450K bead chip type2 design probes into statistical distribution characteristics of type1 probes in order to make their statistical distributions comparable. We then further applied several filtering criteria to reduce the number of CpG methylation probes taken forward for analysis:
a) Remove probes on X or Y chromosome;
b) Remove probes with known SNPs residing in the probe sequence;
c) Remove probes with SNP within 10 bp of the CpG site;
d) Remove non-variable CpG probes if: beta<0.1or beta>0.9 across all samples.
   After these steps, 226047 out of 485577 CpG methylation probes remained. Bioconductor package LIMMA (linear models for microarray data) was used for CpG site-level differential methylation analysis. Out of 226047 sites, 704 CpG sites showed >1.5 fold or <−1.5 fold differential methylation and were significant by LIMMA's moderated t-test (p<0.05) between dry mouth subjects (n=20) and healthy subjects (n=20). 522 CpG sites were differentially hypermethylated in dry mouth parotid gland, while 182 CpG sites were differentially hypomethylated in dry mouth parotid gland. The majority of the 704 differentially methylated sites were located in the gene bodies (45.5%) and within 1500 bp of the transcription start site (TSS1500, 14.3%). 10.8% were located in TSS200 and 7.5% in the 5′UTR region. PCA plot and clustering analysis showed poor separation of DNA methylation between dry mouth and healthy groups (FIGS. 3 and 4). Volcano plot is shown in FIG. 3. Principal component analysis (PCA) plot was used to visualize the separation of the two groups based on expression profiles of the 704 sites (FIG. 4).

In parotid tissues, 704 differentially methylated CpG sites showing significant alterations were found by DNA methylation assay and 167 probes were differentially expressed based on RNA microarrays. DNA hypermethylation is related to gene suppression and hypomethylation to gene expression (Li et al. Front Physiol. 2017; 8:261). The correlation of DNA methylation and RNA transcription of genes identified in this study was examined. By correlating the mRNA expression profiles of the 167 probes with the corresponding methylation profiles and calculating Pearson's correlation coefficients, a list of 14 unique genes with significant negative correlation (i.e., Pearson's correlation<0 and p-value<0.05) between mRNA expression profile and methylation profile was generated. By correlating the methylation expression profiles of the 704 CpG sites with the corresponding mRNA profiles and calculating the Pearson's correlation coefficients, a list of 18 unique genes with significant negative correlation (i.e., Pearson's correlation<0 and p-value<0.05) between mRNA expression profile and methylation profile was generated. The fold changes of expression and DNA methylation of the 14 and 18 genes are shown in Table 1 and 2, respectively. The positive or negative FC values mean the up and down-regulation in dry mouth subjects over healthy subjects, respectively.

To characterize the role of genes associated with the differentially methylated sites, gene ontology (GO) enrichment analysis was performed (Table 3). The gene ontology analysis showed the enrichment of some of these significant genes in biological processes (BP) such as GO:0060075˜regulation of resting membrane potential, GO:0010107˜potassium ion import, GO:0060333˜interferon-gamma-mediated signaling pathway, GO:0015467˜G-protein activated inward rectifier potassium channel activity, and GO:0005242˜inward rectifier potassium channel activity. Genes such as HLA-DQB2 and HLA-F play a role in GO:0060333˜interferon-gamma-mediated signaling pathway. Interferon regulated genes such as MX1 are hypomethylated as seen previously with Sjogren's syndrome and this gene was suggested as a potential biomarker for disease activity and type I interferon bioactivity in Sjogren's syndrome (Ibáñez-Cabellos et al. Front Genet. 2019; 10:1104; Imgenberg-Kreuz et al. Ann Rheum Dis. 2016; 75:2029-36). KEGG pathway analysis using the 18 genes identified 2 genes (KCNJ10 and KCNJ2) that affect the gastric acid secretion pathway by altering potassium transport in and out of the cells (Table 3). It revealed the function of KCNJ10 and KCNJ2 in gastric acid secretion (p=0.052).

TABLE 1
mRNA expression and DNA methylation of 14 genes in parotid tissues

| | | | mRNA Expression | | | DNA methylation | | |
|---|---|---|---|---|---|---|---|---|
| | | p- | (DM vs. H) | | | (DM vs. H) | | |
| Gene | | value. | | Fold | p- | | Fold | p- |
| symbol | Gene Accession | Pearson | Probe | Change | value | CpG site | Change | value |
| OR2B11 | NM_001004492 | 0.039 | TC01004072.hg.1 | −1.31 | 0.030 | cg21302594 | 1.43 | 0.033 |
| HDLBP | NM_005336 | 0.032 | TC02002949.hg.1 | −1.35 | 0.008 | cg17240976 | 1.08 | 0.903 |
| HDLBP | NM_005336 | 0.018 | TC02004902.hg.1 | −1.37 | 0.006 | cg17240976 | 1.08 | 0.903 |
| DHX16 | NM_003587 | 0.030 | TC06003577.hg.1 | −1.36 | 0.004 | cg26951554 | 1.14 | 0.364 |
| C7orf66 | NM_001024607 | 0.019 | TC07001753.hg.1 | 1.31 | 0.047 | cg21462681 | 1.09 | 0.527 |
| SLC25A16 | NM_001324312 | 0.042 | TC10002684.hg.1 | −1.34 | 0.010 | cg10590909 | 1.23 | 0.072 |
| DDX6 | NM_004397 | 0.047 | TC11003376.hg.1 | −1.32 | 0.042 | cg10983623 | 1.12 | 0.189 |
| PNN | NM_002687 | 0.040 | TC14001663.hg.1 | −1.39 | 0.021 | cg18648343 | 1.01 | 0.903 |
| LGALS3 | NM_002306 | 0.023 | TC14001693.hg.1 | −1.82 | 0.002 | cg26335127 | 1.21 | 0.071 |
| USP31 | NM_020718 | 0.010 | TC16001454.hg.1 | 1.31 | 0.023 | cg02052410 | −1.11 | 0.738 |
| USP31 | NM_020718 | 0.018 | TC16001454.hg.1 | 1.31 | 0.023 | cg04453792 | −1.11 | 0.594 |
| TNFAIP8L1 | NM_152362 | 0.048 | TC19001947.hg.1 | −1.41 | 0.007 | cg23662927 | 1.26 | 0.115 |
| ZFR2 | NM_015174 | 0.034 | TC19002296.hg.1 | −1.31 | 0.020 | cg10000139 | 1.05 | 0.677 |
| EMR1 | NM_001256252 | 0.025 | TC19002307.hg.1 | −1.39 | 0.035 | cg22889448 | −1.08 | 0.652 |
| UNC13A | NM_001080421 | 0.003 | TC19002372.hg.1 | 1.39 | 0.001 | cg22989649 | −1.45 | 0.002 |
| MX1 | NM_001144925 | 0.028 | TC21000739.hg.1 | −1.43 | 0.006 | cg15925792 | −1.06 | 0.628 |

TABLE 2
mRNA expression and DNA methylation of 18 genes in parotid tissues

| | | | mRNA Expression | | | DNA methylation | | |
|---|---|---|---|---|---|---|---|---|
| | | | (DM vs. H) | | | (DM vs. H) | | |
| Gene | | p-value | | Fold | p- | | Fold | p- |
| symbol | Gene Accession | Pearson | Probe | change | value | CpG site | change | value |
| PRAMEF20 | NM_001099852 | 0.044 | TC01000179.hg.1 | 1.02 | 0.817 | cg21410132 | 1.52 | 0.025 |
| CCBL2 | NM_001008661 | 0.049 | TC01002844.hg.1 | 1.06 | 0.222 | cg23627354 | −1.55 | 0.041 |
| KCNJ10 | NM_002241 | 0.030 | TC01003396.hg.1 | 1.12 | 0.063 | cg06978270 | 3.10 | 0.021 |
| FAM160A1 | NM_001109977 | 0.036 | TC04000754.hg.1 | 1.09 | 0.091 | cg17479576 | 2.07 | 0.041 |
| KCNIP4 | NM_147182 | 0.034 | TC04002484.hg.1 | 1.09 | 0.434 | cg04974130 | −1.53 | 0.009 |
| HLA-F | NM_001098478 | 0.035 | TC06000323.hg.1 | −1.03 | 0.431 | cg15018934 | 1.58 | 0.047 |
| PSORSIC1 | NM_014068 | 0.046 | TC06000357.hg.1 | 1.03 | 0.577 | cg08412936 | −2.12 | 0.043 |
| ZFAND3 | NM_021943 | 0.019 | TC06000549.hg.1 | −1.02 | 0.752 | cg03985459 | 1.59 | 0.001 |
| KIF25 | NM_030615 | 0.038 | TC06001185.hg.1 | −1.12 | 0.018 | cg09545394 | 1.51 | 0.002 |
| EPHA7 | NM_004440 | 0.006 | TC06001952.hg.1 | −1.07 | 0.124 | cg19464419 | 2.53 | 0.049 |
| HLA- | NR_003937 | 0.030 | TC06003414.hg.1 | −1.05 | 0.684 | cg12296550 | −1.64 | 0.041 |
| DQB2 | | | | | | | | |
| GPR123 | NM_001083909 | 0.039 | TC10000948.hg.1 | −1.03 | 0.519 | cg15731752 | 1.52 | 0.012 |
| SRP54 | NM_003136 | 0.028 | TC14000219.hg.1 | −1.04 | 0.332 | cg04980793 | 2.10 | 0.013 |
| KCNJ2 | NM_000891 | 0.028 | TC17000813.hg.1 | 1.06 | 0.589 | cg12204395 | 1.62 | 0.005 |
| ALOX12B | NM_001139 | 0.042 | TC17001100.hg.1 | 1.00 | 0.940 | cg19437868 | 1.69 | 0.013 |
| KIAA0355 | NM_014686 | 0.033 | TC19000441.hg.1 | −1.07 | 0.097 | cg10087081 | 1.89 | 0.013 |
| SERTAD3 | NM_203344 | 0.031 | TC19001541.hg.1 | 1.04 | 0.569 | cg14150973 | −1.70 | 0.018 |
| PSORSIC1 | NM_014068 | 0.023 | TC6_cox_ | 1.09 | 0.190 | cg08412936 | −2.12 | 0.043 |
| | | | hap2000054.hg.1 | | | | | |
| HLA-F | NM_001098478 | 0.014 | TC6_dbb_ | −1.02 | 0.558 | cg15018934 | 1.58 | 0.047 |
| | | | hap3000016.hg.1 | | | | | |
| PSORSIC1 | NM_014068 | 0.009 | TC6_dbb_ | 1.08 | 0.217 | cg08412936 | −2.12 | 0.043 |
| | | | hap3000047.hg.1 | | | | | |
| HLA-F | NM_001098478 | 0.014 | TC6_mann_ | −1.02 | 0.558 | cg15018934 | 1.58 | 0.047 |
| | | | hap4000017.hg.1 | | | | | |
| PSORSIC1 | NM_014068 | 0.035 | TC6_mann_ | 1.10 | 0.102 | cg08412936 | −2.12 | 0.043 |
| | | | hap4000048.hg.1 | | | | | |
| MICB | NM_005931 | 0.001 | TC6_mann_ | 1.17 | 0.023 | cg23003117 | −1.60 | 0.002 |
| | | | hap4000216.hg.1 | | | | | |
| HLA-F | NM_001098478 | 0.047 | TC6_mcf_ | −1.08 | 0.155 | cg15018934 | 1.58 | 0.047 |
| | | | hap5000203.hg.1 | | | | | |
| HLA-F | NM_001098478 | 0.014 | TC6_qbl_ | −1.02 | 0.558 | cg15018934 | 1.58 | 0.047 |
| | | | hap6000015.hg.1 | | | | | |

TABLE 3
Enriched biological processes for the identified 18 (A, B, C) and 14 (D, E, F) genes
and KEGG pathways in parotid (only significant and up to top 5 terms shown)

| A. GO BP Term | P Value | Genes | Benjamini |
|---|---|---|---|
| GO: 0060075~regulation of resting | 0.004 | KCNJ10, KCNJ2 | 0.32 |
| membrane potential | | | |
| GO: 0010107~potassium ion import | 0.018 | KCNJ10, KCNJ2 | 0.593 |
| GO: 0061337~cardiac conduction | 0.029 | KCNJ2, KCNIP4 | 0.619 |
| GO: 0060333~interferon-gamma-mediated | 0.046 | HLA-DQB2, HLA-F | 0.681 |
| signaling pathway | | | |
| B. GO CC Term | P Value | Genes | Benjamini |
| GO: 0005886~plasma membrane | 0.012 | KCNIP4, KCNJ10, KCNJ2, HLA-DQB2, | 0.34 |
| | | HLA-F, MICB, EPHA7 | |
| GO: 0071556~integral component of lumenal | 0.016 | HLA-DQB2, HLA-F | 0.34 |
| side of endoplasmic reticulum membrane | | | |
| GO: 0012507~ER to Golgi transport vesicle | 0.028 | HLA-DQB2, HLA-F | 0.404 |
| membrane | | | |
| GO: 0008076~voltage-gated potassium | 0.047 | KCNJ2, KCNIP4 | 0.508 |
| channel complex | | | |
| C. GO MF Term | P Value | Genes | Benjamini |
| GO: 0015467~G-protein activated inward | 0.006 | KCNJ10, KCNJ2 | 0.235 |
| rectifier potassium channel activity | | | |
| GO: 0005242~inward rectifier potassium | 0.123 | KCNJ10, KCNJ2 | 0.235 |
| channel activity | | | |
| D. GO BP Term | P Value | Genes | Benjamini |
| GO: 0008380~RNA splicing | 0.095 | LGALS3, DHX16 | 0.999 |
| E. GO CC Term | P Value | Genes | Benjamini |
| GO: 0005886~plasma membrane | 0.095 | HDLBP, EMR1, LGALS3, OR2B11, | 0.999 |
| | | UNC13A, PNN | |
| F. GO MF Term | P Value | Genes | Benjamini |
| GO: 0003724~RNA helicase activity | 0.021 | DHX16, DDX6 | 0.73 |
| GO: 0070035~purine NTP-dependent | 0.069 | DHX16, DDX6 | 0.891 |
| helicase activity | | | |
| GO: 0008026~ATP-dependent helicase | 0.069 | DHX16, DDX6 | 0.891 |
| activity | | | |
| GO: 0017111~nucleoside-triphosphatase | 0.095 | DHX16, MX1, DDX6 | 0.874 |
| activity | | | |
| GO: 0004386~helicase activity | 0.097 | DHX16, DDX6 | 0.795 |
| G. KEGG Pathway Term | P Value | Genes | Benjamini |
| hsa04971: Gastric acid secretion | 0.052 | KCNJ10, KCNJ2 | 0.6892 |

### RNA Profiling and DNA Methylation in Saliva

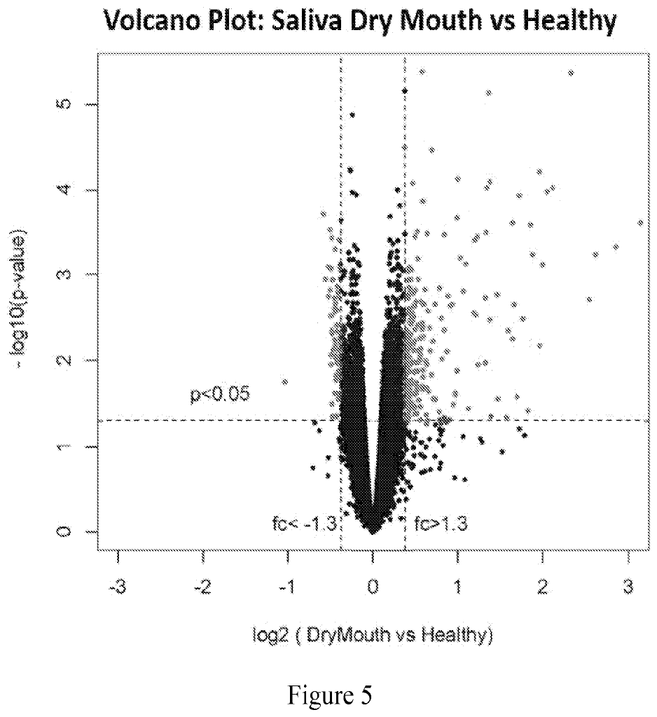

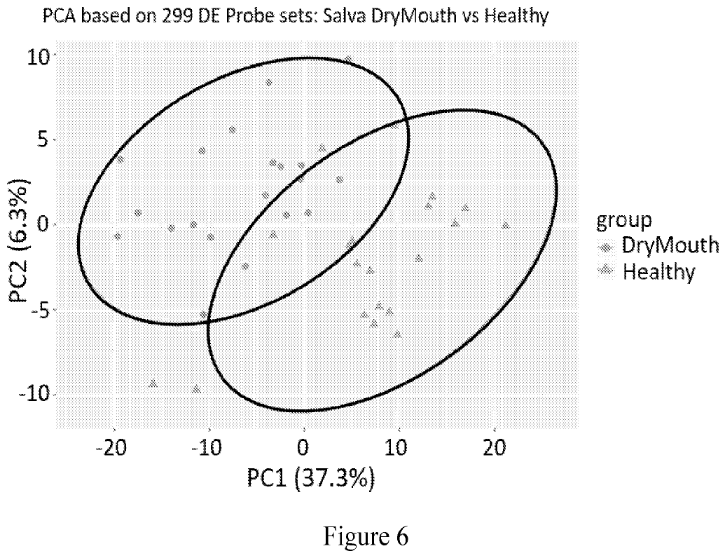

For RNA profiling, RNA was extracted from saliva and quality of the extracted RNA was analyzed by Agilent Bioanalyzer using the RNA 6000 Pico kit as well as the Quant-iTribogreen RNA assay. All 20 healthy and 20 dry mouth saliva samples showed excellent quality and quantity RNA as revealed by the presence of intact 18S and 28S rRNA as well as total RNA yield of >5 ng. The extracted RNA from healthy saliva and dry mouth saliva were constructed for long and small RNA libraries, for a total of 40 libraries. The quality of the RNA libraries were excellent as revealed by long RNA library showing major peak at 300-400 bp whereas small RNA library showing major peak at 140-200 bp. RNA quantity as shown by Qubit dsDNA BR assay revealed concentration >10nm in each sample. The 40 RNA libraries were profiled using the GeneChip Human Transcriptome Affymetrix HTA 2.0 expression arrays. For the analysis of Affymetrix GeneChip HTA 2.0 RNA expression datasets, the Robust Multi-Array Average (RMA) method was applied for background correction. Data were normalized with quantile normalization and Tukey's Median Polish Approach was used to summarize probe intensities. In this step, the measured signal intensities of >6 million probes were summarized into gene level probe sets (n=70523). ComBat method was applied to remove the batch effects of microarrays. We selected probe sets meeting the following criteria:
1) More than 20% arrays have expression index (log2 scale) of at least 5. This step eliminates probes with low expression index;
2) Coefficient of variation is greater than 0.1 across all arrays. This step excludes probes with low variability.
   Using these criteria, 23,111 out of 70,523 probes remained after filtering. Bioconductor package LIMMA (linear models for microarray data) was used for differential gene expression analysis. Out of 23,111 probes, 299 probes showed >1.3 fold or <−1.3 fold differential expression and were significant by LIMMA's moderated t-test (p<0.05) between dry mouth subjects (n=20) and healthy subjects (n=20). Volcano plot is shown in FIG. 5. Principal component analysis (PCA) plot was used to visualize the separation of the two groups based on expression profiles of the 299 gene probes (FIG. 6).

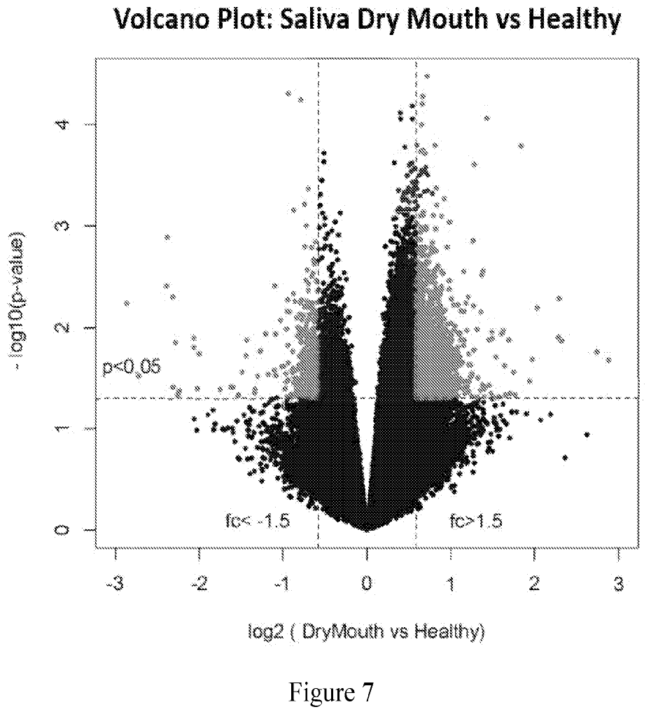

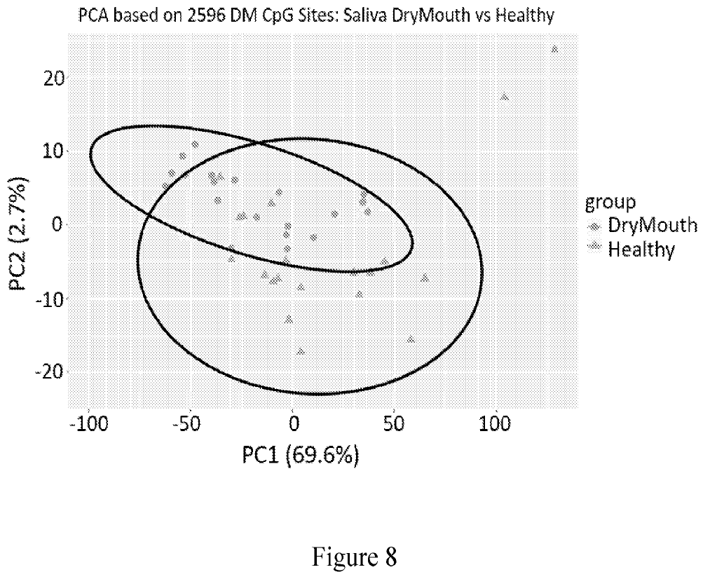

For DNA methylation profiling, DNA was extracted from saliva of 20 healthy and 20 dry mouth subjects using the commercial Pure Link™ Genomic DNA Mini Kit (Life Technologies, Grand Island NY). The concentration of DNA was measured by NanoDrop® ND-1000 Spectrophotometer (Thermo Scientific). The quality of extracted DNA was evaluated by PCR amplification of the housekeeping gene GAPDH (forward primer: TGGTCTGAGGTCTGAGGTTAAAT; reverse primer: TAGTCCCAGGGCTTTGATTTGC). Quality control of the genomic DNA extracted from healthy and dry mouth saliva were all satisfactory as evidenced by the amplification of the 177-bp GAPDH amplicon. Genomic DNAs from healthy and dry mouth saliva were comprehensively profiled using the Illumina human methylation 450K bead chip type2 design probes. For the analysis of Illumina Infinium 450 k DNA methylation datasets, Beta-Mixture Quantile Dilation (BMIQ) Normalization method was applied. This is an intra-sample normalization technique aimed to adjust the beta-values of Illumina human methylation 450K bead chip type2 design probes into statistical distribution characteristics of type1 probes in order to make their statistical distributions comparable. We then further applied several filtering criteria to reduce the number of CpG methylation probes taken forward for analysis:
a) Remove probes on X or Y chromosome;
b) Remove probes with known SNPs residing in the probe sequence;
c) Remove probes with SNP within 10 bp of the CpG site;
d) Remove non-variable CpG probes if: beta<0.1 or beta>0.9 across all samples.
   After these steps, 226047 out of 485577 CpG methylation probes remained. Bioconductor package LIMMA (linear models for microarray data) was used for CpG site-level differential methylation analysis. Out of 226047 sites, 2596 CpG sites related to 1989 genes showed >1.5 fold or <−1.5 fold differential methylation and were significant by LIMMA's moderated t-test (p<0.05) between dry mouth subjects (n=20) and healthy subjects (n=20). 2231 CpG sites were differentially hypermethylated in dry mouth and healthy parotid gland, while 365 CpG sites were differentially hypomethylated in dry mouth and healthy parotid gland. The majority of the 2596 differentially methylated sites were located in the gene bodies (54.5%) and within 1500 bp of the transcription start site (TSS1500, 12.4%). 5.2% were located in TSS200, 4.8% in the 3′UTR region and 8.1% in the 5′UTR region. Volcano plot is shown in FIG. 7. Principal component analysis (PCA) plot was used to visualize the separation of the two groups based on methylation profiles of the 2596 sites (FIG. 8).

In saliva samples, 2596 differentially methylated CpG sites were found by DNA methylation assay and 299 differentially expressed probes were found by RNA microarrays. The correlation of DNA methylation and RNA transcription of genes identified in this study was examined. By correlating the methylation expression profiles of the 2596 CpG sites with the corresponding mRNA profiles and calculating the Pearson's correlation coefficients, a list of 36 unique genes with significant negative correlation (i.e., Pearson's correlation<0 AND p-value<0.05) between mRNA expression profile and methylation profile was generated. By correlating the mRNA expression profiles of the 299 probes with the corresponding methylation profiles and calculating Pearson's correlation coefficients, a list of 61 unique genes with significant negative correlation (i.e., Pearson's correlation<0 and p-value<0.05) between mRNA expression profile and methylation profile was generated. The fold changes of expression and DNA methylation of the 36 and 61 genes are shown in Tables 4 and 5, respectively. The positive or negative FC values mean the up and down-regulation in dry mouth subjects over healthy subjects, respectively.

To characterize the role of genes associated with the differentially methylated sites, gene ontology (GO) enrichment analysis was performed (Table 6). Gene ontology analysis suggested the involvement of a few of these genes in the macromolecular metabolic process and developmental process, among others. KEGG pathway analysis suggested 7 of these 97 genes affecting non-small cell lung cancer (PRKCA, PIK3CG, RASSF5), phosphatidylinositol signaling system (PRKCA, PIK3CG, CDS1), leukocyte transendothelial migration (PRKCA, PIK3CG, RASSF5), and antigen presentation and processing pathways (IFI30, HLA-B, and B2M) (Table 6). RASSF5, IFI30, HLA-B, and B2M have medium expression in the salivary gland (https://www.proteinatlas.org). PRKCA is hypermethylated and downregulated in dry mouth. RASSF5, PIK3CG, IFI30, HLA-B, and B2M are hypomethylated and upregulated. Some of the identified genes such as B2M, TNFAIP3, IFI30, HLA-B, HLA-DR are consistently differentially regulated in Sjogren's syndrome, and B2M is validated as a potential biomarker (Aqrawi et al. Arthritis Res Ther. 2019; 21:181; Nezos et al. J Immunol Res. 2015; 2015:754825). PSORS1C1 gene is differentially expressed both in parotid tissue and saliva. While the corresponding CpG site is hypermethylated in parotid tissue, it is hypomethylated in saliva.

TABLE 4
mRNA expression and DNA methylation of 36 genes in saliva

| | | | mRNA Expression | | | DNA methylation | | |
|---|---|---|---|---|---|---|---|---|
| | | | (DM vs. H) | | | (DM vs. H) | | |
| Gene | Gene | p-value | | Fold | p- | | Fold | p- |
| symbol | Accession | Pearson | Probe | change | value | CpG site | change | value |
| MNDA | NM_002432 | 0.022 | TC01001346.hg.1 | 2.60 | 0.003 | cg05304729 | −1.26 | 0.236 |
| FCER1G | NM_004106 | 0.020 | TC01001382.hg.1 | 1.66 | 0.028 | cg05659526 | −1.15 | 0.154 |
| FCER1G | NM_004106 | 0.028 | TC01001382.hg.1 | 1.66 | 0.028 | cg26394055 | −1.15 | 0.600 |
| BTG2 | NM_006763 | 0.045 | TC01001685.hg.1 | 1.59 | 0.004 | cg00567854 | −1.11 | 0.634 |
| BTG2 | NM_006763 | 0.000 | TC01001685.hg.1 | 1.59 | 0.004 | cg00860712 | −1.25 | 0.159 |
| GOS2 | NM_015714 | 0.047 | TC01001749.hg.1 | 2.53 | 0.000 | cg07434244 | −1.24 | 0.114 |
| GOS2 | NM_015714 | 0.026 | TC01004998.hg.1 | 1.83 | 0.008 | cg07434244 | −1.24 | 0.114 |
| C1orf200 | NR_027045 | 0.013 | TC01005257.hg.1 | 1.40 | 0.007 | cg00231528 | 1.49 | 0.228 |
| C1orf200 | NR_027045 | 0.005 | TC01005257.hg.1 | 1.40 | 0.007 | cg08854008 | −1.18 | 0.533 |
| C1orf200 | NR_027045 | 0.010 | TC01005257.hg.1 | 1.40 | 0.007 | cg11334709 | −1.17 | 0.200 |
| C1orf200 | NR_027045 | 0.030 | TC01005257.hg.1 | 1.40 | 0.007 | cg12354861 | −1.67 | 0.057 |
| C1orf200 | NR_027045 | 0.029 | TC01005257.hg.1 | 1.40 | 0.007 | cg14989202 | 1.09 | 0.844 |
| C1orf200 | NR_027045 | 0.045 | TC01005257.hg.1 | 1.40 | 0.007 | cg16597045 | −1.16 | 0.488 |
| C1orf200 | NR_027045 | 0.011 | TC01005257.hg.1 | 1.40 | 0.007 | cg18284427 | 1.32 | 0.175 |
| C1orf200 | NR_027045 | 0.041 | TC01005257.hg.1 | 1.40 | 0.007 | cg22595920 | −1.41 | 0.316 |
| TAGLN2 | NM_003564 | 0.014 | TC01005883.hg.1 | 1.41 | 0.042 | cg15641364 | −1.04 | 0.698 |
| PLEK | NM_002664 | 0.005 | TC02000398.hg.1 | 1.50 | 0.004 | cg02861056 | −1.23 | 0.267 |
| PLEK | NM_002664 | 0.011 | TC02000398.hg.1 | 1.50 | 0.004 | cg04872689 | −1.45 | 0.187 |
| PLEK | NM_002664 | 0.014 | TC02000398.hg.1 | 1.50 | 0.004 | cg10812236 | −1.53 | 0.157 |
| PLEK | NM_002664 | 0.014 | TC02000398.hg.1 | 1.50 | 0.004 | cg13060970 | −1.26 | 0.230 |
| PLEK | NM_002664 | 0.045 | TC02000398.hg.1 | 1.50 | 0.004 | cg13468685 | −1.27 | 0.293 |
| IL1B | NM_000576 | 0.004 | TC02002219.hg.1 | 2.51 | 0.000 | cg15836722 | −1.20 | 0.368 |
| FXR1 | NM_001013438 | 0.042 | TC03002679.hg.1 | 1.35 | 0.013 | cg01816191 | −1.06 | 0.816 |
| BASP1 | NM_006317 | 0.003 | TC05000096.hg.1 | 1.30 | 0.000 | cg00263146 | −1.22 | 0.018 |
| ATOX1 | NM_004045 | 0.038 | TC05003314.hg.1 | −1.33 | 0.002 | cg21164886 | 1.13 | 0.282 |
| LST1 | NM_001166538 | 0.018 | TC06000372.hg.1 | 1.36 | 0.011 | cg03739609 | −1.32 | 0.464 |
| LST1 | NM_001166538 | 0.004 | TC06000372.hg.1 | 1.36 | 0.011 | cg04398060 | 1.03 | 0.722 |
| LST1 | NM_001166538 | 0.006 | TC06000372.hg.1 | 1.36 | 0.011 | cg09761080 | −1.21 | 0.421 |
| LST1 | NM_001166538 | 0.012 | TC06000372.hg.1 | 1.36 | 0.011 | cg14324675 | −1.32 | 0.274 |
| LST1 | NM_001166538 | 0.006 | TC06000372.hg.1 | 1.36 | 0.011 | cg16795830 | −1.20 | 0.403 |
| LST1 | NM_001166538 | 0.007 | TC06000372.hg.1 | 1.36 | 0.011 | cg19271190 | −1.22 | 0.466 |
| LST1 | NM_001166538 | 0.018 | TC06000372.hg.1 | 1.36 | 0.011 | cg27616007 | −2.10 | 0.049 |
| MARCKS | NM_023009 | 0.040 | TC06000909.hg.1 | 1.38 | 0.006 | cg27397465 | −1.02 | 0.895 |
| HCG22 | NR_003948 | 0.026 | TC06002705.hg.1 | −1.34 | 0.003 | cg11039913 | 1.33 | 0.231 |
| LST1 | NM_001166538 | 0.047 | TC06002715.hg.1 | 1.72 | 0.039 | cg03739609 | −1.32 | 0.464 |
| LST1 | NM_001166538 | 0.014 | TC06002715.hg.1 | 1.72 | 0.039 | cg04398060 | −1.03 | 0.722 |
| LST1 | NM_001166538 | 0.012 | TC06002715.hg.1 | 1.72 | 0.039 | cg09761080 | −1.21 | 0.421 |
| LST1 | NM_001166538 | 0.021 | TC06002715.hg.1 | 1.72 | 0.039 | cg14324675 | −1.32 | 0.274 |
| LST1 | NM_001166538 | 0.013 | TC06002715.hg.1 | 1.72 | 0.039 | cg16795830 | −1.20 | 0.403 |
| LST1 | NM_001166538 | 0.012 | TC06002715.hg.1 | 1.72 | 0.039 | cg19271190 | 1.22 | 0.466 |
| LST1 | NM_001166538 | 0.012 | TC06002715.hg.1 | 1.72 | 0.039 | cg27616007 | −2.10 | 0.049 |
| RPS18 | NM_022551 | 0.022 | TC06002737.hg.1 | 1.96 | 0.024 | cg09591519 | −1.23 | 0.065 |
| RPS18 | NM_022551 | 0.013 | TC06002737.hg.1 | 1.96 | 0.024 | cg15484808 | −1.10 | 0.171 |
| MARCKS | NM_023009 | 0.015 | TC06003006.hg.1 | 1.33 | 0.012 | cg27397465 | −1.02 | 0.895 |
| TNFAIP3 | NM_024309 | 0.009 | TC06003084.hg.1 | 1.35 | 0.018 | cg05987705 | −1.03 | 0.830 |
| BTNL2 | NM_019602 | 0.028 | TC06003406.hg.1 | 1.50 | 0.004 | cg02591634 | −1.04 | 0.712 |
| ABO | NM_020469 | 0.008 | TC09002317.hg.1 | 1.46 | 0.010 | cg12020464 | −1.08 | 0.770 |
| PFKFB3 | NM_001145443 | 0.049 | TC10001860.hg.1 | 1.76 | 0.009 | cg00872580 | −1.12 | 0.557 |
| PFKFB3 | NM_001145443 | 0.020 | TC10001860.hg.1 | 1.76 | 0.009 | cg16179674 | −1.31 | 0.214 |
| PFKFB3 | NM_001145443 | 0.002 | TC10001860.hg.1 | 1.76 | 0.009 | cg18989491 | 1.59 | 0.036 |
| PFKFB3 | NM_001145443 | 0.000 | TC10001860.hg.1 | 1.76 | 0.009 | cg22692545 | −1.27 | 0.014 |
| PFKFB3 | NM_001145443 | 0.019 | TC10001860.hg.1 | 1.76 | 0.009 | cg26262157 | −1.38 | 0.275 |
| PFKFB3 | NM_001145443 | 0.003 | TC10001860.hg.1 | 1.76 | 0.009 | cg27545615 | −1.48 | 0.092 |
| MIR708 | NR_030598 | 0.027 | TC11002142.hg.1 | −1.30 | 0.008 | cg05473648 | 1.08 | 0.513 |
| FTH1 | NM_002032 | 0.036 | TC11003179.hg.1 | 3.02 | 0.005 | cg21421501 | −1.16 | 0.265 |
| PFDN5 | NM_002624 | 0.044 | TC12000435.hg.1 | 1.38 | 0.010 | cg07661836 | −1.02 | 0.863 |
| LYZ | NM_000239 | 0.002 | TC12000611.hg.1 | 1.35 | 0.013 | cg16097772 | −1.68 | 0.011 |
| RPS29 | NM_001032 | 0.040 | TC14001098.hg.1 | 1.51 | 0.022 | cg11188516 | 1.20 | 0.149 |
| RPS29 | NM_001032 | 0.024 | TC14001098.hg.1 | 1.51 | 0.022 | cg27175475 | −1.04 | 0.790 |
| B2M | NM_004048 | 0.015 | TC15000342.hg.1 | 4.32 | 0.000 | cg18696027 | −1.32 | 0.175 |
| MIR548H4 | NR_031680 | 0.003 | TC15000631.hg.1 | −2.04 | 0.017 | cg08413060 | 1.05 | 0.586 |
| MIR548H4 | NR_031680 | 0.005 | TC15000631.hg.1 | −2.04 | 0.017 | cg09727046 | 1.06 | 0.517 |
| MIR548H4 | NR_031680 | 0.014 | TC15000631.hg.1 | −2.04 | 0.017 | cg22381808 | 1.23 | 0.019 |
| B2M | NM_004048 | 0.044 | TC15002179.hg.1 | 8.89 | 0.000 | cg18696027 | −1.57 | 0.159 |
| TMEM186 | NM_015421 | 0.042 | TC16000850.hg.1 | 1.30 | 0.018 | cg00011459 | 1.01 | 0.942 |
| LITAF | NM_001136472 | 0.002 | TC16000870.hg.1 | 1.34 | 0.002 | cg08767044 | −1.59 | 0.066 |
| LITAF | NM_001136472 | 0.045 | TC16000870.hg.1 | 1.34 | 0.002 | cg09160589 | −1.14 | 0.266 |
| LITAF | NM_001136472 | 0.036 | TC16001773.hg.1 | 1.39 | 0.002 | cg03071793 | −2.41 | 0.017 |
| LITAF | NM_001136472 | 0.003 | TC16001773.hg.1 | 1.39 | 0.002 | cg08767044 | −1.59 | 0.066 |
| LITAF | NM_001136472 | 0.013 | TC16001773.hg.1 | 1.39 | 0.002 | cg09160589 | −1.14 | 0.266 |
| ARRB2 | NM_004313 | 0.003 | TC17000047.hg.1 | 1.35 | 0.001 | cg02286380 | −1.23 | 0.465 |
| ARRB2 | NM_004313 | 0.001 | TC17000047.hg.1 | 1.35 | 0.001 | cg07971820 | −1.28 | 0.171 |
| ARRB2 | NM_004313 | 0.001 | TC17000047.hg.1 | 1.35 | 0.001 | cg13466002 | 1.31 | 0.417 |
| ARRB2 | NM_004313 | 0.001 | TC17000047.hg.1 | 1.35 | 0.001 | cg16472369 | −1.48 | 0.270 |
| ARRB2 | NM_004313 | 0.003 | TC17000047.hg.1 | 1.35 | 0.001 | cg19265289 | −1.29 | 0.478 |
| GABARAP | NR_028287 | 0.042 | TC17001081.hg.1 | 1.32 | 0.031 | cg10230466 | 1.17 | 0.216 |
| MIR512- | NR_030181 | 0.042 | TC19000815.hg.1 | −1.41 | 0.001 | cg11978784 | 1.30 | 0.010 |
| 1 | | | | | | | | |
| MIR512- | NR_030181 | 0.042 | TC19000816.hg.1 | −1.41 | 0.001 | cg11978784 | 1.30 | 0.010 |
| 1 | | | | | | | | |
| GMFG | NM_004877 | 0.008 | TC19001517.hg.1 | 2.50 | 0.011 | cg05607401 | −1.10 | 0.639 |
| MYO1F | NM_012335 | 0.022 | TC19002321.hg.1 | 1.34 | 0.029 | cg11667738 | 1.04 | 0.730 |
| MYO1F | NM_012335 | 0.008 | TC19002321.hg.1 | 1.34 | 0.029 | cg26269802 | −1.33 | 0.104 |
| MYO1F | NM_012335 | 0.011 | TC19002321.hg.1 | 1.34 | 0.029 | cg27582235 | −1.18 | 0.295 |
| IFI30 | NM_006332 | 0.021 | TC19002629.hg.1 | 1.41 | 0.002 | cg26152923 | −1.06 | 0.756 |
| TTPAL | NM_001039199 | 0.025 | TC20001231.hg.1 | −1.32 | 0.007 | cg25750259 | 1.01 | 0.965 |
| ATP5O | NM_001697 | 0.006 | TC21000938.hg.1 | 1.42 | 0.024 | cg15249164 | −1.04 | 0.640 |
| HLA-B | NM_005514 | 0.004 | TC6_qbl_ | 1.92 | 0.002 | cg03500977 | −1.02 | 0.840 |
| | | | hap6000148.hg.1 | | | | | |
| HLA-B | NM_005514 | 0.002 | TC6_qbl_ | 1.92 | 0.002 | cg15454374 | −1.11 | 0.422 |
| | | | hap6000148.hg.1 | | | | | |
| RPS18 | NM_022551 | 0.018 | TC6_ssto_ | 1.43 | 0.018 | cg09591519 | −1.23 | 0.065 |
| | | | hap7000088.hg.1 | | | | | |

TABLE 5
mRNA expression and DNA methylation of 61 genes in saliva

| | | | mRNA Expression | | | DNA methylation | | |
|---|---|---|---|---|---|---|---|---|
| | | | (DM vs. H) | | | (DM vs. H) | | |
| Gene | Gene | p-value | | Fold | p- | | Fold | p- |
| symbol | Accession | Pearson | Probe | change | value | CpG site | change | value |
| PARK7 | NM_007262 | 0.019 | TC01000101.hg.1 | −1.02 | 0.570 | cg12446447 | 1.76 | 0.010 |
| TFAP2E | NM_178548 | 0.032 | TC01000459.hg.1 | 1.06 | 0.342 | cg18131141 | −1.55 | 0.011 |
| TFAP2E | NM_178548 | 0.024 | TC01000459.hg.1 | 1.06 | 0.342 | cg24685006 | −1.56 | 0.007 |
| PPIH | NM_006347 | 0.043 | TC01000529.hg.1 | −1.09 | 0.054 | cg04482817 | 1.54 | 0.032 |
| RASSF5 | NM_182663 | 0.028 | TC01001724.hg.1 | 1.15 | 0.025 | cg22401939 | −1.55 | 0.040 |
| RERE | NM_012102 | 0.019 | TC01005249.hg.1 | −1.20 | 0.082 | cg19679865 | 1.72 | 0.003 |
| PEF1 | NM_012392 | 0.030 | TC01005382.hg.1 | −1.00 | 0.966 | cg11955456 | 1.51 | 0.026 |
| ARHGAP15 | NM_018460 | 0.030 | TC02000904.hg.1 | 1.13 | 0.015 | cg19867914 | −1.83 | 0.001 |
| THUMPD3 | NM_015453 | 0.011 | TC03000037.hg.1 | 1.04 | 0.235 | cg06679221 | −1.69 | 0.008 |
| EOMES | NM_005442 | 0.008 | TC03001257.hg.1 | −1.00 | 0.964 | cg20739013 | −1.60 | 0.049 |
| PLSCR5 | NM_001085420 | 0.045 | TC03001870.hg.1 | 1.02 | 0.708 | cg22594071 | 1.87 | 0.017 |
| CDS1 | NM_001263 | 0.043 | TC04000462.hg.1 | 1.00 | 0.927 | cg22884714 | 1.52 | 0.037 |
| SYNPO2 | NM_001128934 | 0.042 | TC04002167.hg.1 | 1.18 | 0.179 | cg11569478 | 1.84 | 0.035 |
| HOPX | NM_139212 | 0.037 | TC04002559.hg.1 | 1.05 | 0.529 | cg06771126 | 1.65 | 0.028 |
| ANKRD33B | NM_001164440 | 0.049 | TC05000077.hg.1 | 1.01 | 0.828 | cg16343302 | 1.55 | 0.010 |
| FBXL7 | NM_012304 | 0.049 | TC05000088.hg.1 | −1.03 | 0.569 | cg19641327 | 1.73 | 0.005 |
| RNF14 | NM_183399 | 0.016 | TC05000776.hg.1 | −1.01 | 0.775 | cg04182865 | −1.53 | 0.039 |
| PSORSIC1 | NM_014068 | 0.019 | TC06000357.hg.1 | −1.02 | 0.630 | cg20564865 | 1.86 | 0.041 |
| LST1 | NR_029461 | 0.018 | TC06000372.hg.1 | 1.36 | 0.011 | cg27616007 | −2.10 | 0.049 |
| RUNX2 | NM_001015051 | 0.050 | TC06000621.hg.1 | 1.07 | 0.070 | cg22456162 | −1.62 | 0.004 |
| PRDM1 | NM_001198 | 0.021 | TC06000844.hg.1 | 1.04 | 0.325 | cg08358263 | 1.57 | 0.017 |
| MOG | NM_206813 | 0.020 | TC06002367.hg.1 | −1.08 | 0.427 | cg16118803 | 1.51 | 0.041 |
| LST1 | NR_029461 | 0.027 | TC06002437.hg.1 | 1.18 | 0.044 | cg27616007 | −2.10 | 0.049 |
| LST1 | NR_029461 | 0.012 | TC06002715.hg.1 | 1.72 | 0.039 | cg27616007 | −2.10 | 0.049 |
| HLA- | NR_001435 | 0.035 | TC06002733.hg.1 | −1.06 | 0.470 | cg03943025 | 2.05 | 0.006 |
| DPB2 | | | | | | | | |
| TNXB | NM_019105 | 0.029 | TC06003375.hg.1 | −1.11 | 0.233 | cg16735938 | 1.53 | 0.020 |
| TNXB | NM_019105 | 0.026 | TC06003376.hg.1 | −1.14 | 0.064 | cg11608893 | 1.50 | 0.006 |
| TNXB | NM_032470 | 0.014 | TC06003381.hg.1 | −1.16 | 0.104 | cg06819251 | 3.40 | 0.047 |
| SYCP2L | NM_001040274 | 0.009 | TC06004048.hg.1 | −1.06 | 0.094 | cg13351621 | 1.64 | 0.024 |
| TAP2 | NM_000544 | 0.017 | TC06004126.hg.1 | −1.09 | 0.059 | cg08998192 | 1.50 | 0.016 |
| GRM3 | NM_000840 | 0.042 | TC07000519.hg.1 | 1.03 | 0.463 | cg01331810 | 1.60 | 0.023 |
| BUD31 | NM_003910 | 0.005 | TC07000593.hg.1 | 1.10 | 0.048 | cg18634506 | −1.54 | 0.017 |
| PIK3CG | NM_002649 | 0.028 | TC07000687.hg.1 | 1.11 | 0.005 | cg11982525 | −1.53 | 0.022 |
| C7orf50 | NM_001134395 | 0.032 | TC07001077.hg.1 | 1.03 | 0.523 | cg12692727 | 1.80 | 0.048 |
| C7orf50 | NM_001134395 | 0.022 | TC07001077.hg.1 | 1.03 | 0.523 | cg15086474 | 1.54 | 0.039 |
| RBM28 | NM_018077 | 0.041 | TC07001844.hg.1 | 1.04 | 0.292 | cg24107852 | −1.79 | 0.047 |
| KIAA1147 | NM_001080392 | 0.030 | TC07001930.hg.1 | 1.03 | 0.558 | cg06390077 | 1.64 | 0.007 |
| MSR1 | NM_138715 | 0.034 | TC08002237.hg.1 | 1.07 | 0.317 | cg16303562 | −1.55 | 0.015 |
| PFKFB3 | NM_001145443 | 0.000 | TC10000053.hg.1 | 1.22 | 0.004 | cg18989491 | −1.59 | 0.036 |
| ADK | NM_001123 | 0.020 | TC10000479.hg.1 | −1.01 | 0.785 | cg11717883 | 1.87 | 0.045 |
| SH2D4B | NM_207372 | 0.027 | TC10000583.hg.1 | 1.02 | 0.631 | cg10374402 | 1.80 | 0.039 |
| PFKFB3 | NM_001145443 | 0.002 | TC10001860.hg.1 | 1.76 | 0.009 | cg18989491 | −1.59 | 0.036 |
| LRRC27 | NM_001143757 | 0.022 | TC10002376.hg.1 | 1.00 | 0.948 | cg07119315 | −1.68 | 0.006 |
| PTDSS2 | NM_030783 | 0.011 | TC11000017.hg.1 | −1.10 | 0.015 | cg00554604 | 1.90 | 0.008 |
| PTDSS2 | NM_030783 | 0.013 | TC11000017.hg.1 | −1.10 | 0.015 | cg16197643 | 1.58 | 0.036 |
| ALDH3B2 | NM_001031615 | 0.039 | TC11002740.hg.1 | −1.14 | 0.260 | cg16338278 | 1.74 | 0.029 |
| ARHGEF17 | NM_014786 | 0.035 | TC11002781.hg.1 | 1.26 | 0.002 | cg03499808 | 1.75 | 0.033 |
| EFEMP2 | NM_016938 | 0.011 | TC11003210.hg.1 | 1.04 | 0.378 | cg17616283 | −1.55 | 0.025 |
| MMP3 | NM_002422 | 0.023 | TC11003313.hg.1 | −1.25 | 0.031 | cg16466334 | 1.64 | 0.010 |
| LYZ | NM_000239 | 0.002 | TC12000611.hg.1 | 1.35 | 0.013 | cg16097772 | −1.68 | 0.011 |
| SLC17A8 | NM_001145288 | 0.035 | TC12000776.hg.1 | −1.08 | 0.095 | cg06563300 | 1.60 | 0.016 |
| MGP | NM_000900 | 0.032 | TC12001276.hg.1 | 1.07 | 0.405 | cg00431549 | 1.61 | 0.036 |
| MGP | NM_000900 | 0.011 | TC12001276.hg.1 | 1.07 | 0.405 | cg05360958 | −1.51 | 0.037 |
| BICD1 | NM_001714 | 0.016 | TC12002306.hg.1 | 1.12 | 0.314 | cg15075784 | 1.72 | 0.009 |
| MGP | NM_000900 | 0.026 | TC12002778.hg.1 | 1.07 | 0.515 | cg00431549 | −1.61 | 0.036 |
| MGP | NM_000900 | 0.009 | TC12002778.hg.1 | 1.07 | 0.515 | cg05360958 | −1.51 | 0.037 |
| RTN1 | NM_021136 | 0.034 | TC14001182.hg.1 | −1.02 | 0.625 | cg10829004 | 1.53 | 0.044 |
| GCNT3 | NM_004751 | 0.045 | TC15000456.hg.1 | 1.13 | 0.025 | cg00437411 | 1.84 | 0.038 |
| GCNT3 | NM_004751 | 0.048 | TC15000456.hg.1 | −1.13 | 0.025 | cg01247856 | 1.77 | 0.047 |
| GALNS | NM_000512 | 0.003 | TC16001345.hg.1 | 1.06 | 0.115 | cg26817641 | −1.58 | 0.022 |
| LITAF | NR_024320 | 0.036 | TC16001773.hg.1 | 1.39 | 0.002 | cg03071793 | −2.41 | 0.017 |
| ERI2 | NM_001142725 | 0.017 | TC16002040.hg.1 | −1.07 | 0.195 | cg06579338 | 1.56 | 0.038 |
| PRKCA | NM_002737 | 0.014 | TC17000783.hg.1 | −1.08 | 0.058 | cg00498360 | 1.72 | 0.034 |
| PRKCA | NM_002737 | 0.043 | TC17000783.hg.1 | 1.08 | 0.058 | cg14343701 | 1.76 | 0.034 |
| PRKCA | NM_002737 | 0.011 | TC17000783.hg.1 | 1.08 | 0.058 | cg24171047 | 1.71 | 0.010 |
| C17orf59 | NM_017622 | 0.001 | TC17001108.hg.1 | 1.00 | 0.994 | cg02067584 | −1.84 | 0.026 |
| ST6GALNAC1 | NM_018414 | 0.013 | TC17001904.hg.1 | 1.02 | 0.744 | cg00440980 | 2.00 | 0.049 |
| GFAP | NM_001131019 | 0.018 | TC17002230.hg.1 | −1.18 | 0.064 | cg09639715 | 1.75 | 0.038 |
| ACACA | NM_198837 | 0.020 | TC17002601.hg.1 | −1.06 | 0.396 | cg20778688 | 1.54 | 0.026 |
| ZNF772 | NM_001024596 | 0.007 | TC19001896.hg.1 | −1.02 | 0.545 | cg23391173 | 1.78 | 0.011 |
| RIN2 | NM_018993 | 0.034 | TC20000134.hg.1 | 1.02 | 0.552 | cg03416521 | 1.81 | 0.045 |
| ITSN1 | NM_003024 | 0.005 | TC21000683.hg.1 | −1.21 | 0.079 | cg07708472 | 1.53 | 0.006 |
| COL18A1 | NM_130445 | 0.005 | TC21000793.hg.1 | 1.07 | 0.386 | cg01212562 | 1.91 | 0.046 |
| COL18A1 | NM_130445 | 0.007 | TC21000793.hg.1 | −1.07 | 0.386 | cg01314574 | 1.59 | 0.011 |
| CECR1 | NM_017424 | 0.050 | TC22001480.hg.1 | −1.11 | 0.011 | cg10714773 | 1.51 | 0.017 |
| TAP2 | NM_000544 | 0.016 | TC6_apd_ | −1.10 | 0.067 | cg08998192 | 1.50 | 0.016 |
| | | | hap1000130.hg.1 | | | | | |
| PSORSIC1 | NM_014068 | 0.019 | TC6_cox_ | −1.06 | 0.229 | cg20564865 | −1.60 | 0.049 |
| | | | hap2000054.hg.1 | | | | | |
| TAP2 | NM_000544 | 0.023 | TC6_cox_ | −1.09 | 0.037 | cg08998192 | 1.50 | 0.016 |
| | | | hap2000257.hg.1 | | | | | |
| TAP2 | NM_000544 | 0.016 | TC6_dbb_ | −1.10 | 0.067 | cg08998192 | 1.50 | 0.016 |
| | | | hap3000242.hg.1 | | | | | |
| TNXB | NM_032470 | 0.011 | TC6_mann_ | −1.09 | 0.027 | cg06819251 | 3.40 | 0.047 |
| | | | hap4000149.hg.1 | | | | | |
| HLA- | NM_002124 | 0.022 | TC6_mann_ | −1.10 | 0.191 | cg11404906 | 3.12 | 0.024 |
| DRB1 | | | hap4000158.hg.1 | | | | | |
| TAP2 | NM_000544 | 0.014 | TC6_mann_ | −1.09 | 0.054 | cg08998192 | 1.50 | 0.016 |
| | | | hap4000165.hg.1 | | | | | |
| HLA- | NM_002124 | 0.022 | TC6_mcf_ | −1.10 | 0.191 | cg11404906 | 3.12 | 0.024 |
| DRB1 | | | hap5000168.hg.1 | | | | | |
| TAP2 | NM_000544 | 0.028 | TC6_mcf_ | −1.07 | 0.137 | cg08998192 | 1.50 | 0.016 |
| | | | hap5000225.hg.1 | | | | | |
| TAP2 | NM_000544 | 0.015 | TC6_qbl_ | −1.08 | 0.062 | cg08998192 | 1.50 | 0.016 |
| | | | hap6000185.hg.1 | | | | | |
| HLA- | NM_002124 | 0.022 | TC6_ssto_ | −1.10 | 0.191 | cg11404906 | 3.12 | 0.024 |
| DRB1 | | | hap7000162.hg.1 | | | | | |
| TAP2 | NM_000544 | 0.016 | TC6_ssto_ | −1.10 | 0.067 | cg08998192 | 1.50 | 0.016 |
| | | | hap7000213.hg.1 | | | | | |

TABLE 6
Enriched biological processes for the identified 36 (A, B, C) and 61 (D, E, F) genes
and KEGG pathways in saliva (only significant and up to top 5 terms shown)

| A. GO BP Term | P value | Genes | Benjamini |
|---|---|---|---|
| GO: 0048002~antigen processing and | 0 | IFI30, FCER1G, HLA-B, B2M | 0.015 |
| presentation of peptide antigen | | | |
| GO: 0002478~antigen processing and | 0 | IFI30, FCER1G, B2M | 0.068 |
| presentation of exogenous peptide | | | |
| antigen | | | |
| GO: 0019884~antigen processing and | 0 | IFI30, FCER1G, B2M | 0.075 |
| presentation of exogenous antigen | | | |
| GO: 0002376~immune system | 0 | LST1, PLEK, IFI30, FCER1G, IL1B, MYOIF, | 0.071 |
| process | | HLA-B, FTH1, B2M | |
| GO: 0019882~antigen processing and | 0.001 | IFI30, FCER1G, HLA-B, B2M | 0.074 |
| presentation | | | |
| B. GO CC Term | P value | Genes | Benjamini |
| GO: 0044444~cytoplasmic part | 0 | LST1, LITAF, PLEK, PFKFB3, ATOX1, | 0.003 |
| | | MYO1F, IFI30, GABARAP, FTH1, B2M, | |
| | | FXR1, RPS18, TMEM186, RPS29, ARRB2, | |
| | | PFDN5, IL1B, MARCKS, ATP50, TNFAIP3, | |
| | | ABO | |
| GO: 0005737~cytoplasm | 0.004 | LST1, LITAF, PLEK, PFKFB3, ATOX1, IFI30, | 0.221 |
| | | MYOIF, GABARAP, FTH1, B2M, FXR1, | |
| | | RPS18, TMEM186, RPS29, ARRB2, PFDN5, | |
| | | MNDA, IL1B, MARCKS, ATP50, TNFAIP3, | |
| | | ABO | |
| GO: 0005829~cytosol | 0.03 | RPS18, PLEK, RPS29, ATOX1, PFKFB3, | 0.756 |
| | | PFDN5, FTH1 | |
| GO: 0005622~intracellular | 0.03 | LST1, LITAF, ATOX1, PFKFB3, IFI30, | 0.655 |
| | | TAGLN2, FTH1, B2M, RPS29, IL1B, ATP5O, | |
| | | PLEK, MYO1F, BASP1, GABARAP, FXR1, | |
| | | TTPAL, TMEM186, RPS18, ARRB2, GMFG, | |
| | | PFDN5, MNDA, MARCKS, TNFAIP3, ABO | |
| GO: 0044445~cytosolic part | 0.031 | RPS18, RPS29, PFDN5 | 0.582 |
| C. GO BP Term | P value | Genes | Benjamini |
| GO: 0044259~multicellular | 0.005 | TNXB, ACACA, MMP3 | 0.971 |
| organismal macromolecule | | | |
| metabolic process | | | |
| GO: 0044236~multicellular | 0.006 | TNXB, ACACA, MMP3 | 0.918 |
| organismal metabolic process | | | |
| GO: 0051094~positive regulation of | 0.013 | PRKCA, MSR1, HOPX, EOMES, RUNX2 | 0.962 |
| developmental process | | | |
| GO: 0050793~regulation of | 0.021 | PRKCA, MSR1, LST1, HOPX, EOMES, MGP, | 0.985 |
| developmental process | | RUNX2 | |
| GO: 0051216~cartilage development | 0.024 | PRKCA, MGP, RUNX2 | 0.978 |
| D. GO CC Term | P value | Genes | Benjamini |
| GO: 0005622~intracellular | 0.002 | GCNT3, GFAP, LST1, LITAF, PFKFB3, | 0.233 |
| | | SYCP2L, ERI2, ARHGAP15, MMP3, ITSN1, | |
| | | RTN1, BUD31, PEF1, TAP2, GALNS, ZNF772, | |
| | | RBM28, RUNX2, RNF14, PTDSS2, PIK3CG, | |
| | | PRKCA, TNXB, ACACA, EOMES, CECR1, | |
| | | MGP, ARHGEF17, SYNPO2, CDS1, PARK7, | |
| | | BICD1, ST6GALNAC1, SLC17A8, RASSF5, | |
| | | GRM3, PPIH, ADK, HOPX, RIN2, FBXL7, | |
| | | PRDM1, TFAP2E, RERE | |
| GO: 0044424~intracellular part | 0.016 | GCNT3, GFAP, LST1, LITAF, PFKFB3, | 0.644 |
| | | SYCP2L, ARHGAP15, MMP3, ITSN1, RTN1, | |
| | | BUD31, PEF1, TAP2, GALNS, ZNF772, | |
| | | RBM28, RUNX2, RNF14, PIK3CG, PRKCA, | |
| | | EOMES, CECR1, ACACA, MGP, ARHGEF17, | |
| | | SYNPO2, CDS1, PARK7, BICD1, | |
| | | ST6GALNAC1, SLC17A8, RASSF5, GRM3, | |
| | | PPIH, ADK, HOPX, RIN2, FBXL7, PRDM1, | |
| | | TFAP2E, RERE | |
| GO: 0005578~proteinaceous | 0.017 | COL18A1, TNXB, EFEMP2, MGP, MMP3 | 0.505 |
| extracellular matrix | | | |
| GO: 0005615~extracellular space | 0.018 | COL18A1, MSR1, TNXB, LYZ, CECR1, MGP, | 0.434 |
| | | MMP3 | |
| GO: 0031012~extracellular matrix | 0.021 | COL18A1, TNXB, EFEMP2, MGP, MMP3 | 0.418 |
| E. GO MF Term | P value | Genes | Benjamini |
| GO: 0005201~extracellular matrix | 0.003 | COL18A1, TNXB, EFEMP2, MGP | 0.504 |
| structural constituent | | | |
| GO: 0017169~CDP-alcohol | 0.027 | CDS1, PTDSS2 | 0.951 |
| phosphatidyltransferase activity | | | |
| GO: 0005088~Ras guanyl-nucleotide | 0.037 | RIN2, ARHGEF17, ITSN1 | 0.935 |
| exchange factor activity | | | |
| GO: 0016780~phosphotransferase | 0.06 | CDS1, PTDSS2 | 0.966 |
| activity, for other substituted | | | |
| phosphate groups | | | |
| GO: 0005198~structural molecule | 0.066 | COL18A1, GFAP, TNXB, EFEMP2, MGP, | 0.949 |
| activity | | BICD1 | |
| F. KEGG Pathway | P value | Genes | Benjamini |
| hsa05223: Non-small cell lung cancer | 0.015 | PRKCA, PIK3CG, RASSF5 | 0.697 |
| hsa04070: Phosphatidylinositol | 0.028 | PRKCA, PIK3CG, CDS1 | 0.663 |
| signaling system | | | |
| hsa04612: Antigen processing and | 0.021 | IFI30, HLA-B, B2M | 0.484 |
| presentation | | | |

Medication-induced dry mouth is an important geriatric problem that requires intervention to improve the quality of life. By adopting a data-driven bioinformatic approach, we have identified the cellular and mechanistic signatures that might be unique to dry mouth. Some of the identified genes and pathways have a strong relationship to Sjogren's syndrome, which indicate a possible similarity in the pathophysiology of both conditions. The findings of this study will enable specific targeting for diagnostic and personalized treatment strategy of dry mouth.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1. A method of diagnosing xerostomia in a subject, comprising:
(a) isolating a biological sample from the subject;
(b) detecting a level of expression and/or DNA methylation of at least one gene selected from genes listed in Tables 1, 2, 4, and 5 in the saliva sample from the subject;
(c) comparing the level of expression and/or DNA methylation of the at least one gene in the sample to a level of expression and/or DNA methylation in a reference,
wherein an increased or decreased level of expression and/or DNA methylation of the at least one gene in the sample compared to the level in the reference identifies the subject having xerostomia and wherein the biological sample is biopsied parotid gland or saliva.

2. The method of claim 1, wherein the at least one gene is selected from the group consisting of KCNJ10, KCNJ2, PRKCA, PIK3CG, RASSF5, CDS1, IFI30, HLA-B, and B2M.

3. The method of claim 1, wherein the at least one gene comprises KCNJ10 and KCNJ2.

4. The method of claim 1, wherein the at least one gene comprises PRKCA, PIK3CG, RASSF5, CDS1, IFI30, HLA-B, and B2M.

5. The method of claim 1, wherein the at least one gene comprises PRKCA, PIK3CG, RASSF5.

6. The method of claim 1, wherein the at least one gene comprises PRKCA, PIK3CG, CDS1.

7. The method of claim 1, wherein the at least one gene comprises IFI30, HLA-B, and B2M.

8. The method of claim 1, wherein the level of expression of the at least one gene in the biological sample is determined by measuring the level of mRNA of the at least one gene in the biological sample.

9. The method of claim 1, wherein the level of expression of the at least one gene in the biological sample is determined by measuring the level of polypeptide of the at least one gene in the biological sample.

10. The method of claim 1, wherein the level of DNA methylation of the at least one gene in the biological sample is determined by measuring the level of DNA methylation at a CpG site located within or near the gene, optionally wherein the CpG site is located in the promoter region of the gene.

11. A method of treating a subject suffering from xerostomia, comprising:
(a) diagnosing xerostomia according to claim 1, and
(b) administering a xerostomia treatment to the subject.

12. The method of claim 11, wherein the treatment comprises administering a administering a therapeutic agent (e.g. pilocarpine) that boosts saliva production to the subject, applying an oral care composition containing an agent to treat or alleviate xerostomia or reduce friction between oral surfaces or boost salivary production (e.g., an oral care composition comprising a fluoride ion source, artificial saliva substitute or moisturizers, or a mouthwash such as Colgate® Hydris™ Oral Rinse) to the oral cavity, and changing medications that causes xerostomia (e.g., adjusting the dose of medication or switching to a different drug that doesn't cause xerostomia) if the subject has taken medications that causes xerostomia or a combination thereof.

13. A method of monitoring the response to a xerostomia treatment in a subject, comprising
(a) isolating a biological sample from the subject after the treatment is initiated;
(b) detecting a level of expression and/or DNA methylation of at least one gene selected from genes listed in Tables 1, 2, 4, and 5 in the biological sample from the subject;
(c) comparing the level of expression and/or DNA methylation of the at least one gene in the sample to a level of expression and/or DNA methylation in a reference,
wherein an increased or decreased level of expression and/or DNA methylation of the at least one gene in the sample compared to the level in the reference indicates that the subject is responsive to the treatment and wherein the biological sample is biopsied parotid gland or saliva.

14. A kit for diagnosing and/or monitoring xerostomia, comprising at least one reagent for the determination of the level of mRNA or polypeptide or the level of DNA methylation of at least one gene selected from genes listed in Tables 1, 2, 4, and 5.

15. The kit of claim 14, wherein the kit comprises at least one reagent for the determination of the level of mRNA of the at least one gene, optionally wherein the at least one reagent comprises amplification primer pairs (forward and reverse) and/or probes specific for the mRNA of interest.

16. The kit of claim 14, wherein the kit comprises at least one reagent for the determination of the level of polypeptide of the at least one gene, optionally wherein the at least one reagent comprises monoclonal antibodies specific for the polypeptide of interest.

17. The kit of claim 14, wherein the kit comprises at least one reagent for the determination of the level of DNA methylation of the at least one gene, optionally wherein the at least one reagent comprises a pair of oligonucleotides (e.g., oligonucleotides attached to two different bead types) specific for the methylated and unmethylated DNA site (e.g., CpG site) of interest.

18. The method of claim 1, wherein said detecting step (b) comprises obtaining a biological sample of a subject and detecting a level of expression (e.g., mRNA or polypeptide) and/or DNA methylation of the at least one gene in the biological sample of the subject, wherein the level of mRNA of the at least one gene is detected by nucleic acid microarrays, quantitative PCR, real time PCR, sequencing (e.g., next generation sequencing), or the level of polypeptide of the at least one gene is detected by ELISA, Western blot, flow cytometry, immunofluorescence, immunohistochemistry, and mass spectroscopy, or the level of DNA methylation of the at least one gene is detected by bisulfite sequencing, methylation specific melting curve analysis (MS-MCA), high resolution melting (MS-HRM), MALDI-TOF MS, methylation specific MLPA, methylated-DNA precipitation/enrichment and methylation-sensitive restriction enzymes (COMPARE-MS), methylation sensitive oligonucleotide microarray, Infinium and MethylLight via antibodies and protein binding domains targeted to methylated DNA or single molecule real time sequencing, Multiplex methylation based PCR assays, Illumina Methylation Assay using ‘BeadChip’ technology, and wherein the biological sample is biopsied parotid gland or saliva.

19. The method of claim 13, wherein said detecting step (b) comprises obtaining a biological sample of a subject and detecting a level of expression (e.g., mRNA or polypeptide) and/or DNA methylation of the at least one gene in the biological sample of the subject, wherein the level of mRNA of the at least one gene is detected by nucleic acid microarrays, quantitative PCR, real time PCR, sequencing (e.g., next generation sequencing), or the level of polypeptide of the at least one gene is detected by ELISA, Western blot, flow cytometry, immunofluorescence, immunohistochemistry, and mass spectroscopy, or the level of DNA methylation of the at least one gene is detected by bisulfite sequencing, methylation specific melting curve analysis (MS-MCA), high resolution melting (MS-HRM), MALDI-TOF MS, methylation specific MLPA, methylated-DNA precipitation/enrichment and methylation-sensitive restriction enzymes (COMPARE-MS), methylation sensitive oligonucleotide microarray, Infinium and MethylLight via antibodies and protein binding domains targeted to methylated DNA or single molecule real time sequencing, Multiplex methylation based PCR assays, Illumina Methylation Assay using ‘BeadChip’ technology, and wherein the biological sample is biopsied parotid gland or saliva.
